SEQUENCE LISTING (6 sequences)

SEQ ID NO: 1 - DNA, 716 bp, Artificial Sequence
  VH-linker-VL portion of VK-8-1.9
caggtccagctgcaggagtcaggacctgaactggtgaaacctggggcctcagtgaagatatcctgcaagcttctggatacacattcactgactacaacatgcactgggtgaagcagagccatggaaagagccttgagtggattggatatatttatccttacaatggtgatactggctacaaccagaaattcaggaacaaggcctccttgactgtagacacttcctccagcacagcctacatggagctccgcagcctgacatctgaggactctgcagtctattactgtgcaagatctggggggttttggtacttcgatgtctggggccaagggaccacggtcaccgtctcctcaggtggaggcggttcaggcggaggtggctctggcggtggcggatcggacatccagatgacccagtctccagcaatcctgtctgcatctccaggggagaaggtcacaatgacttgcagggccaccccaagtgtaagttacatgcactggtatcagcagaagccaggatcctcccccaaaccttggatttataccacatccaacctggcttctggagtccctgctcgcttcagtggcggtgggtctgggacctcttactctctcacagtcagcagagtggaggctgaagatgctgccacttattactgccagcagtggagtcgtagcccacccacgttcggtgctggcaccaagctggaaataaaa

SEQ ID NO: 2 - DNA, 717 bp, Artificial Sequence
  VH-linker-VL portion of OC125-3.11
caggtgcagctgcagcagtcagggactgtgctggcaaggcctggggcttcagtgaagatgtccttgcaagcttctggctacacctttaccagctactggatgcactgggtaaaacagaggcctggacagggtctggaatggattggcgctatttatcctggaaatagtgatactagctacaaccagaagttcaagggcaaggccaaactgactgcagtcacatccaccagcactgcctacatggagctcagcagcctgacaaatgaggactctgcggtctattactgtacacgtagctacgactggtacttcgatgtctggggccaagggaccacggtcaccgtctcctcaggtggaggcggttcaggcggaggtggctctggcggtggcggatcggacatcgagctcactcagtctccagccatcctgtctgtgagtccaggagaaagagtcagtttctcctgcagggccagtcagagcattggcacagacatgcactggtatcagcaaagaacaaatggttctccaaggcttctcataaagtatgcttctgagtctatctctgggatcccttccaggtttagtggcagtggatcagggacagattttactcttagcatcaacagtgtggagtctgaagatattgcagattattactgtcaacaaagttatagctggccgctcacgttcggtgctgggacaaagttggaaataaaa

SEQ ID NO: 3 - DNA, 329 bp, Artificial Sequence
  VH portion of VK-8-1.9
caggtccagctgcaggagtcaggacctgaactggtgaaacctggggcctcagtgaagatatcctgcaagcttctggatacacattcactgactacaacatgcactgggtgaagcagagccatggaaagagccttgagtggattggatatatttatccttacaatggtgatactggctacaaccagaaattcaggaacaaggcctccttgactgtagacacttcctccagcacagcctacatggagctccgcagcctgacatctgaggactctgcagtctattactgtgcaagatctggggggttttggtacttcgatgtctggggccaa

SEQ ID NO: 4 - DNA, 318 bp, Artificial Sequence
  VL portion of VK-8-1.9
gacatccagatgacccagtctccagcaatcctgtctgcatctccaggggagaaggtcacaatgacttgcagggccaccccaagtgtaagttacatgcactggtatcagcagaagccaggatcctcccccaaaccttggatttataccacatccaacctggcttctggagtccctgctcgcttcagtggcggtgggtctgggacctcttactctctcacagtcagcagagtggaggctgaagatgctgccacttattactgccagcagtggagtcgtagcccacccacgttcggtgctggcaccaagctggaaataaaa

SEQ ID NO: 5 - DNA, 327 bp, Artificial Sequence
  VH portion of OC125-3.11
caggtgcagctgcagcagtcagggactgtgctggcaaggcctggggcttcagtgaagatgtccttgcaagcttctggctacacctttaccagctactggatgcactgggtaaaacagaggcctggacagggtctggaatggattggcgctatttatcctggaaatagtgatactagctacaaccagaagttcaagggcaaggccaaactgactgcagtcacatccaccagcactgcctacatggagctcagcagcctgacaaatgaggactctgcggtctattactgtacacgtagctacgactggtacttcgatgtctggggccaa

SEQ ID NO: 6 - DNA, 321 bp, Artificial Sequence
  VL portion of OC125-3.11
gacatcgagctcactcagtctccagccatcctgtctgtgagtccaggagaaagagtcagtttctcctgcagggccagtcagagcattggcacagacatgcactggtatcagcaaagaacaaatggttctccaaggcttctcataaagtatgcttctgagtctatctctgggatcccttccaggtttagtggcagtggatcagggacagattttactcttagcatcaacagtgtggagtctgaagatattgcagattattactgtcaacaaagttatagctggccgctcacgttcggtgctgggacaaagttggaaataaaa

(19) United States
(12) Patent Application Publication
(10) Pub. No.: US 2006/0134116 A1
(43) Pub. Date: Jun. 22, 2006

(54) Ca 125 tumor antigen function and uses thereof

(75) Inventors: Claudine Rancourt, Sherbrooke (CA); Alain Piche, Sherbrooke (CA); Julie Beaudin, Sherbrooke (CA)

Correspondence Address: MERCHANT & GOULD PC, P.O. BOX 2903, MINNEAPOLIS, MN, 55402-0903, US

(73) Assignee: Universite De Sherbrooke, Sherbrooke, QC (CA)

(21) Appl. No.: 10/507,728

(22) PCT Filed: Mar. 11, 2003
(86) PCT No.: PCT/CA03/00341

§ 371 Date: May 27, 2005

Related U.S. Application Data
(60) Provisional application No. 60/363,306, filed on Mar. 11, 2002.

(30) Foreign Application Priority Data
Feb. 28, 2003 (CA) 2,420,494

(51) Int. Cl. A61K 39/395; A61K 39/00; C07H 21/04; C12P 21/06; C07K 14/82; C07K 16/30

(52) U.S. Cl. 424/155.1; 424/185.1; 435/69.1; 435/320.1; 435/325; 530/350; 536/23.5; 530/388.8

(57) ABSTRACT

The present invention is directed to modulators of CA 125 tumor antigen, a recombinant nucleic acids, vectors, host cells, pharmaceutical compositions, uses of the foregeoing for negatively modulating CA 125 tumor antigen in mammalian cell, preventing and treating a CA 125 tumor antigen associated disease in a mammal, as well as methods of prevention and treatment of such diseases and methods of negatively regulating CA 125 tumor antigen in a mammalian cell.

### FIELD OF THE INVENTION

This invention relates to CA 125 tumor antigen. More specifically, it relates to modulators of CA 125 tumor antigen and to their uses in the treatment and prevention of diseases wherein CA 125 tumor antigen is overexpressed.

### BACKGROUND OF THE INVENTION

Ovarian cancer is one of the leading causes of death in women over 40. Although most patients respond to initial treatment, the majority relapses partially due to the appearance of chemo-resistant tumor cells. In order to improve therapy, it is essential to understand the underlying mechanisms responsible for the occurrence of ovarian cancer.

CA125 Antigen is the Most Important Clinical Marker of Ovarian Cancer

CA125 tumor antigen is the most important clinical marker of ovarian cancer as it is used to monitor response to chemotherapy. Rising or falling blood levels of CA125 correlate with progression or regression of the disease. CA125 antigen was first detected in the early 80's using the MAb OC125 which was raised against the human ovarian carcinoma cell line OV433 isolated from a patient with serous papillary cystadenocarcinoma (1). The specific reactivity of the OC125 Mab to a variety of human ovarian carcinoma cell lines and paraffin-embedded ovarian carcinoma tissues has led to the development of a radioimmunoassay to detect the CA125 antigen in serum from ovarian cancer patients (2). Using this assay, rising or falling levels of CA125 were shown to correlate with progression or regression of disease demonstrating that CA 125 levels correlate with clinical course of the disease (2-4). It is currently employed as a predictor of clinical recurrence in ovarian cancer and to monitor response to chemotherapy treatment (5-8).

CA125 Biochemical Studies

Despite the widespread use of CA125 as a clinical marker of ovarian cancer, the biochemical and molecular nature as well as the function of this antigen are poorly understood. Previous biochemical studies demonstrated that the CA125 epitope is carried on a large glycoprotein with a M.W. in the range of 2×10⁵-10⁶ Da, while others reported that CA125 consists of many subunits of 50-200 kDa (9-14). The study of Lloyd et al. showed that CA125 is a high molecular weight glycoprotein having properties of a mucin-type molecule (15). In these studies however, a definite consensus regarding the molecular nature of CA 125 could not be elaborated and no information about its function was provided. A partial cDNA encoding CA125 was recently identified as MUC16. The deduced amino acid sequence proposed an extracellular domain composed of 9 tandem repeats rich in serine, threonine and proline followed by a unique region, a potential transmembrane domain and a short cytoplasmic tail. CA125 is expressed in more than 80% of epithelial ovarian cancer but is not detectable in normal ovary tissues. However its role in the disease is unknown.

There is therefore a crucial need to identify therapeutic targets in order to treat the disease.

### SUMMARY OF THE INVENTION

An object of the present invention is to provide a therapeutic target that satisfies the above mentioned need.

The present inventors developed a novel strategy to study the role of proteins that could not be previously studied because the gene was not known or not available. Using this strategy, they derived unique modulators of CA125 tumor antigen. The present inventors propose a functional link between CA125 tumor antigen and the pathogenesis of ovarian cancer as well as other diseases where CA 125 tumor antigen is overexpressed, a non exclusive list of which comprises endometriosis, cervical cancer, fallopian tube cancer, cancer of the uterus, prostate cancer and lung cancer. The inventors' results have lead to the identification of CA 125 tumor antigen and CA 125 tumor antigen function as novel therapeutic targets for the treatment and prevention of these diseases in mammals.

Accordingly, an object of the present invention provides for a modulator capable of negatively modulating a CA 125 tumor antigen in a mammalian cell.

According to a prefered aspect of the present invention, the modulator negatively modulates cell surface expression of CA 125 tumor antigen. Preferably, the modulator sequesters CA 125 tumor antigen or a fragment thereof within an organelle of a mammalian cell, such as the endoplasmic reticulum, the trans-golgi, the golgi, the mitochondrion, the cytoplasm or any other cellular compartment.

According to another prefered aspect of the present invention, the modulator is a single-chain antibody that specifically binds to CA 125 tumor antigen or a fragment thereof. The single-chain antibody is preferably derived from the OC 125 monoclonal antibody or VK-8 monoclonal antibody. The single-chain antibody preferably comprises a fragment coded by at least one sequence of the group consisting of SEQ. ID NOS 1 to 6.

According to a second object of the present invention, there is provided a recombinant nucleic acid comprising at least one sequence selected from the group consisting of SEQ ID NOS 1 to 6.

According to a third object of the present invention, there is provided a vector comprising a recombinant nucleic acid as defined by the present invention.

According to a fourth aspect of the present invention, there is provided a host cell comprising at least one element selected from the group consisting of a modulator as defined by the present invention, a recombinant nucleic acid as defined by the present invention and a vector as defined by the present invention.

According to fifth object of the present invention, there is provided a pharmaceutical composition comprising a pharmaceutically acceptable carrier and at least one element selected from the group consisting of a modulator as defined by the present invention, a recombinant nucleic acid as defined by the present invention, a vector as defined by the present invention, and a host cell as defined by the present invention.

According to a sixth object of the present invention, there is provided a use of at least one element selected from the group consisting of a modulator as defined by the present invention, a recombinant nucleic acid as defined by the present invention and a vector as defined by the present invention for negatively modulating a CA 125 tumor antigen in a mammalian cell.

According to a seventh object of the present invention, there is provides a use of at least one element selected from the group consisting of a modulator as defined by the present invention, a recombinant nucleic acid as defined by the present invention, a vector as defined by the present invention, a host cell as defined by the present invention and a pharmaceutical composition as defined by the present invention for preventing and treating a CA 125-tumor-antigen-associated disease in a mammal.

According to an eighth object of the present invention, there is provided a method of prevention or treatment of a CA 125-tumor-antigen-associated disease in a mammal comprising the step of administrating to that mammal at least one element selected from the group consisting of a modulator as defined by the present invention, a recombinant nucleic acid as defined by the present invention, a vector as defined by the present invention, and a host cell as defined by the present invention.

According to a ninth object of the present invention, there is provided a method for negatively modulating a CA 125 tumor antigen in a mammalian cell comprising the step of introducing into that cell at least one element selected from the group consisting of a modulator as defined by the present invention, a recombinant nucleic acid as defined by the present invention, and a vector as defined by the present invention.

Other objects, advantages and features of the present invention will become more apparent upon reading of the following non restrictive description of preferred embodiments thereof, given by way of example only with reference to the accompanying drawings.

### BRIEF DESCRIPTION OF THE DRAWINGS

FIG. 1 shows the proposed structure of the CA 125 tumor antigen.

FIGS. 2A and B show the construction of an ScFv library.

FIGS. 3A and B show the selection of soluble ScFvs through a “colony lift assay”.

FIG. 4 shows selection of soluble ScFvs (periplasmic extrcts)

FIG. 5 shows expression of ScFvs (ELISA).

FIG. 6 shows expression of ScFvs in the pCantab-5E prokaryotic expression system with and without induction.

FIG. 7 shows selection of ScFvs binding to CA 125 (ELISA).

FIG. 8 shows selection of ScFvs binding to CA 125 (ELISA).

FIG. 9 shows cloning of ScFvs binding to CA 125 in eukaryotic expression system.

FIGS. 10A, B, C and D show Western blots showing expression of ScFvs directed to the golgi in OVCAR-3 and directed to the ER in OVCAR-3.

FIG. 11 shows expression of ScFv OC125 golgi 3.11 compared with expression of CA 125.

FIG. 12 show expression of ScFv VK-8 KDEL 1.9 compared with expression of CA 125.

FIG. 13 shows expression of control linker compared with expression of CA 125.

FIG. 14 shows expression of ScFv in golgi and expression of ScFv in ER compared with expression of proteins native to golgi and ER, respectively.

FIG. 15 illustrates construction and in vitro validation of anti-CA125 scFvs.

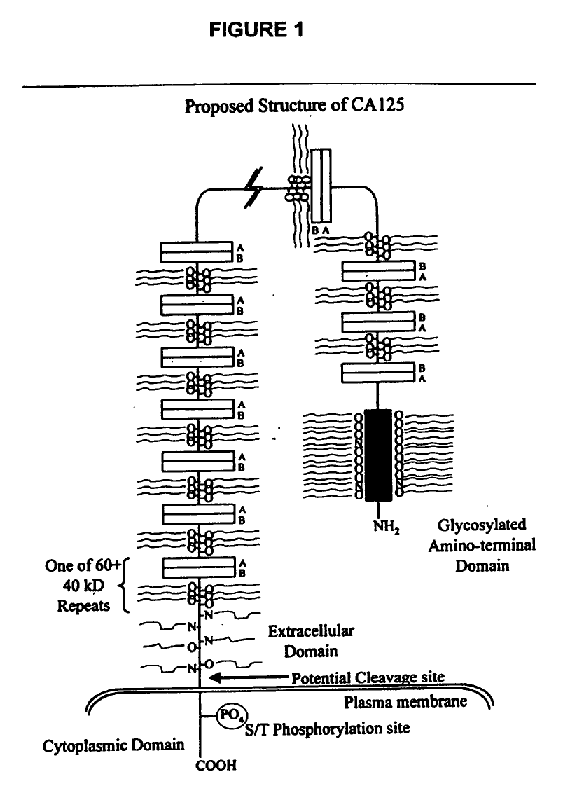

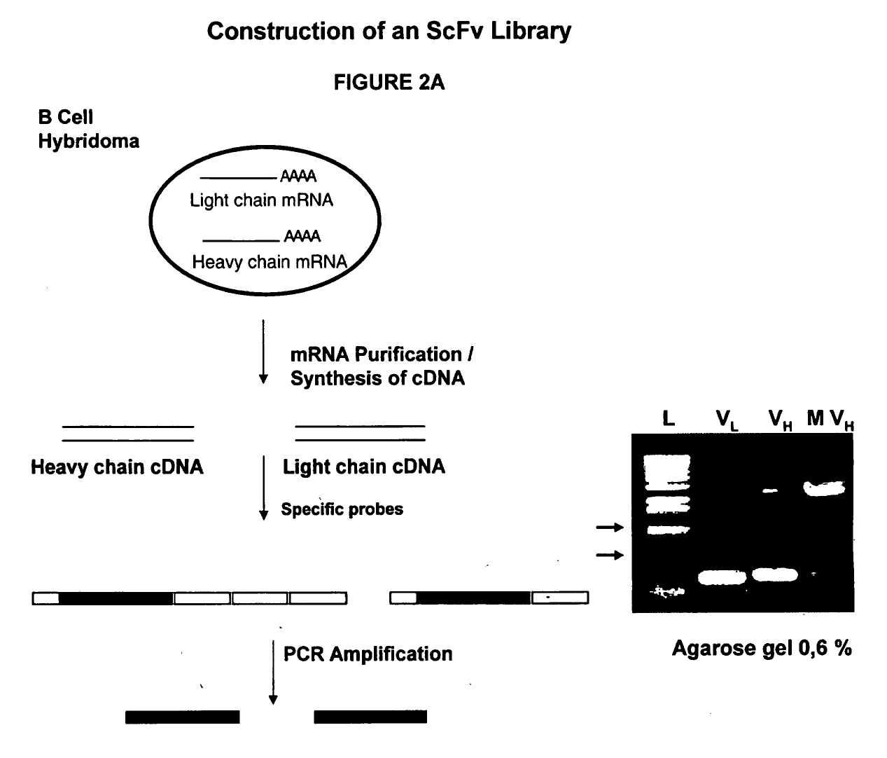

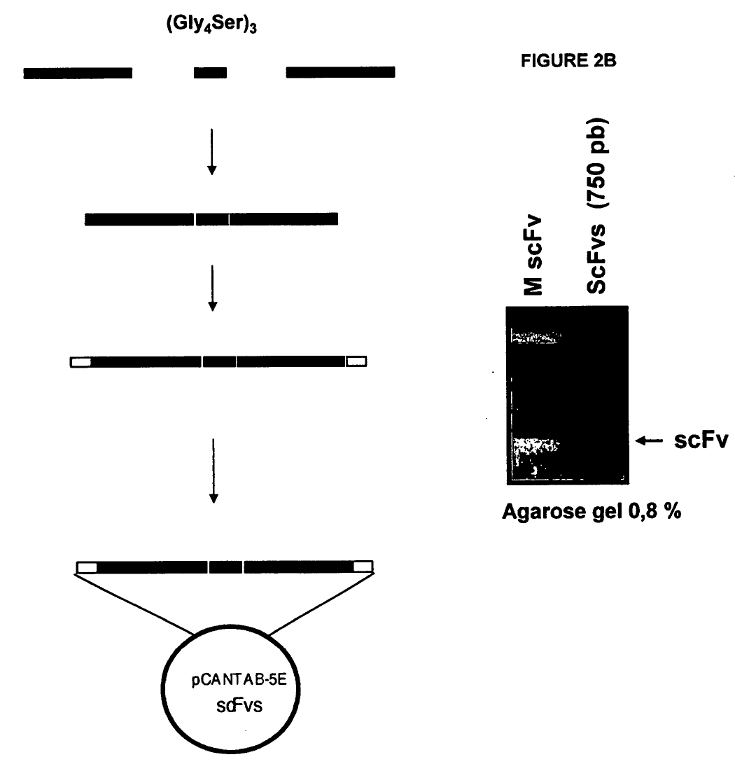

A) CA125 binding activity of anti-CA125 OC125, VK-8-1.9 and VK-8-4.5 scFvs present in periplasmic extracts of bacteria as well as anti-Bcl2 4D7 scFv compared to parental Mabs OC125 and VK-8 and to PBS and periplasmic extract from bacteria, uninduced, IPTG-induced and controls.

B) Expression of scFvs from periplasmic extracts, probed with anti-Etag antibody.

C) Immunoprecipitation and co-immunoprecipitation of Golgi- and ER-targeted OC125-3.11 scFv from transient transfection of pSTCF.GOLGI-OC125-3.11 and pSTCF.KDEL-OC125-3.11 in NIH:OVCAR-3 human ovarian cancer cells using anti-c-myc, anti-CA125 Mabs OC125 and VK-8, western blot probed with anti-c-myc 9E10 antibody.

D) Immunoprecipitation and co-immunoprecipitation of Golgi- and ER-targeted VK-8-1.9 scFv from transient transfection of pSTCF-GOLGI-VK8-1.9 and pSTCF.KDEL-VK8-1.9 in NIH:OVCAR-3 human ovarian cancer cells using anti-c-myc, anti-CA125 Mabs OC125 and VK-8, Western blot probed with anti-c-myc 9E10 antibody.

E) Immunoprecipitation and co-immunoprecipitation of ER-targeted VK-8-4.5 scFv from transient transfection of pSTCF.KDEL-VK8-4.5 in NIH:OVCAR-3 human ovarian cancer cells using anti-c-myc, anti-CA125 Mabs OC125 and VK-8, western blot prober with anti-c-myc 9E10 antibody.

FIG. 16 shows localization of anti-CA125 scFvs and CA125 cell surface down regulation.

A) NIH:OVCAR-3 cells were transiently transfected with pSTCF.Golgi-OC125-3.11 or pSTCF.KDEL-VK-8-1.9 constructs and 48 hrs later the cells were fixed in ice-cold methanol. Localization of scFvs was detected with the anti-c-myc A14 polyclonal antibody and compared with ER and Golgi residents using anti-calreticulin PA3-900 and anti-ADP ribosylation factor MA3-060 monoclonal antibodies, respectively. Oregon green anti-rabbit and texas red anti-Mouse secondary antibodies were used.

B) NIH:OVCAR-3 cellswere transiently transfected with pSTCF.Golgi-OC125-3.11 or pSTCF.KDEL-VK-8-1.9 constructs and 48 hrs later the cells were fixed in ice-cold methanol. Expression of scFvs and CA125 was detected using the anti-c-myc A14 polyclonal antibody and anti-CA125 M11 monoclonal antibody. Oregon green anti-rabbit and Texas red anti-mouse secondary antibodies were used.

FIG. 17 shows Cell surface down modulation of CA125 in stable NIH:OVCAR-3 clones expressing the ER-VK-8-1.9 anti-CA125scFv and relevant control.

A) Stable transfectants expressing the ER-targeted VK-8-1.9 and VK-8-4.5 scFvs and parental cell line NIH:OVCAR-3 were fixed in ice-cold methanol generated and expression of CA125 at the cell surface and scFv was assesed by immunofluorescence using anti-c-myc A14 polyclonal antibody and anti-CA125 M11 monoclonal antibody, respectively. Oregon green anti-rabbit and Texas red anti-mouse antibodies were used as secondary antibodies.

B) CA125 expression in the stable trasnfectants was analysed by FACS using anti-CA125 M11 monoclonal antibody and a Phyco-Erythrin-anti-mouse antibody and compared with parental cell line NIH:OVCAR-3; Black, OVCAR-3 levels of CA125 expression at cell surface; grey, CA125 levels in stable transfectants.

FIG. 18 shows decreased CA125 cell surface expression influences the proliferation rate, cell-cell interaction and cell migration.

A) Growth curve of stable NIH:OVCAR-3 transfectants ER-VK-8-1.9#9 (positive for CA125 binding) and ER-VK-8-4.5#12 (negative for CA125 binding) compared to parental cells OVCAR-3. Cells were plated in triplicate in 96-well plate and cell proliferation was measured every day with a XTT assay. Plot represents results from 3 independent experiments

B) Cell aggregation assay. Cells were plated onto 0.6% agarose layer in bacterial dishes. Seventy-two hours later photomicrographs were taken (10× magnification) to visualize the presence of cell aggregates.

C) Wound healing assay. A wound was made using a 13 mm-wide razor blade in confluent cell monolayers and 20 mM hydroxy-urea was added to block cell proliferation. Forty-eight hours later, the cells were fied in methanol and stained with Giemsa and microphotographs were taken (1OX magnification).

D) Tumorigenic assay. Ten millions NIH:OVCAR-3 transfectants ER-VK-8-1.9#9, ER-VK-8-4.5#12 and parental cells OVCAR-3 were inoculated subcutaneously in nude mice Tumors were allowed to grow for 6 weeks after which tumorw were excised and tumor weight was measured and plotted for each transfectant.

FIG. 18.1 shows results of clonogenic assay.

Increasing amounts of each transfectant, VK-8 KDEU1:9#9 and KDEL/4:5#12, and the parental cell line NIH OVCAR-3 were seeded in 6-well plates and grown in the absence or the presence of doxycycline. Fourteen days later, cells were stained with Giemsa and colonies were scored. Arrows show colonies and numbers correspond to the amount of cells initially seeded. Number of colony formed was plotted against increasing amounts of cells seeded (FIG. 18.1A). Plates were scanned to visualize the size of stained colonies (FIG. 18.1B-D).

FIG. 19

A) NEDO cDNA clone FLJ14303 encodes a part of CA125. Cos-7 cells (negative for CA125 by Western blot and ELISA) were transfected with an expression vector encoding the cDNA from the NEDO clone FLJ14303. Reactivity of anti-CA125 OC125 and VK-8 antibodies with the expression product of this cDNA was analysed by western blot and compared to CA125 expression in OVCAR-3 cells as well as in mock-transfected Cos-7 cells.

B) Expression of the CA125 cytoplasmic tail fused to Gal4 DNA binding domain. The CA125 cytoplasmic tail was cloned in the pGBDU and pGAD for the yeast two-hybrid system. The S. Cerevisiae strain PJ69-4a was transformed with the pGBDU empty vector (EV) or with the vector Containing CA125 cytoplasmic tail (Cyto). Three days after growth on appropriate media, proteins were extracted from the 2 transfectants or the wt strain PJ69-4a, ran on 12.5% SDS-PAGE and transfered by western blot on a PVDF membrane. The membrane was probed with anti-Gal4 DNA binding domain antibody Gal-4-DBD RK5C1.

FIG. 20 shows cisplatin sensibility of stable NIH:OVCAR-3 clones expressing the ER-VK-8-1.9 anti-CA125scFv and relevant controls.

Cells were plated in triplicate in 96-well plates and exposed or not to increasing concentrations of cisplatin. Fours days later, cell proliferation was measured with a XTT assay. Percentage of survival was plotted against concentration of cisplatin. Curves represent results from 3 independent experiments. Red line represents 50% survival.

FIG. 20.1 shows the relative distribution of cells in each phase of the cell cycle for transfectants ERVK-8-1.9#9 and ERVK-8-4.5#12.

DNA content of non-synchronized cells was stained with propidium iodide at various time points for approximately 48 hours. FIGS. 20.1A1 to A4 show the different relative profiles of cell cycle progression: graphs 001 to 007 (ERVK-8-4.5#12) and 008 to 014(ERVK-8-1.9#9) at 0, 8,16, 24, 32, 40 and 48 hrs. FIG. 20.1B is a table form of cell cycle progression and shows the relative percentages of cells in each phase of the cell cycle as determined by FACS analysis.

FIG. 21 shows IC50 of cisplatin for the stable NIH:OVCAR-3 clones expressing the ER-VK-B-1.9 anti-CA125scFv and relevant controls. Inhibitory concentrations of cisplatin resulting in 50% survival of cells were calculated from curves of graph in FIG. 20 (red line in FIG. 20).

FIG. 22 shows expression of E-cadherin and avp5 integrin in NIH:OVCAR-3 cells. NIH:OVCAR-3 were fixed in ice-cold methanol generated and expression of E-cadherin and αvβ5 integrin at the cell surface was assesed by immunofluorescence using E-cadherin clone 36 and anti-αvβ5 integrin clone P1F6 antibody and Texas red labelled secondary anti-mouse antibody.

FIG. 23 shows expression of E-cadherin and scFv in NIH:OVCAR-3 stable transfectant expressing the ER-VK-8-1.9 anti-CA125 scFv without induction with doxycycline. Cells were grown on glass slides for 48 hrs and fixed in ice-cold methanol generated and expression of E-cadherin and scFv was assesed by immunofluorescence using E-cadherin clone 36 and anti-c-myc A14 antibody and Texas red or Oregon green-conjugated secondary anti-mouse and anti-rabbit antibodies.

FIG. 24 shows expression of E-cadherin and scFv in NIH:OVCAR-3 stable transfectant expressing the ER-VK-8-1.9 anti-CA125 scFv when induced with doxycycline. Cells were grown in presence of doxycycline for 48 hrs and then fixed in ice-cold methanol generated and expression of E-cadherin and scFv was assessed by immunofluorescence using E-cadherin clone 36 and anti-c-myc A14 antibody and Texas red or Oregon green-conjugated secondary anti-mouse and anti-rabbit antibodies.

FIG. 25 shows expression of E-cadherin and scFv in NIH:OVCAR-3 stable transfectant expressing the ER-VK-4.5 control scFv without induction with doxycycline. Cells were grown on glass slides for 48 hrs and fixed in ice-cold methanol generated and expression of E-cadherin and scFv was assesed by immunofluorescence using E-cadherin clone 36 and anti-c-myc A14 antibody and Texas red or Oregon green-conjugated secondary anti-mouse and anti-rabbit antibodies.

FIG. 26 shows expression of E-cadherin and scFv in NIH:OVCAR-3 stable transfectant expressing the ER-VK4.5 control scFv when induced with doxycycline. Cells were grown on glass slides in the presence of doxycycline for 48hrs and fixed in ice-cold methanol generated and expression of E-cadherin and scFv was assesed by immunofluorescence using E-cadherin clone 36 and anti-c-myc A14 antibody and Texas red or Oregon green-conjugated secondary anti-mouse and anti-rabbit antibodies.

FIG. 27 shows expression of αvβ5 integrin and scFv in NIH:OVCAR-3 stable transfectant expressing the ER-VK-8-1.9 anti-CA125 scFv when induced or not with doxycycline. Cells were grown in the absence or presence of doxycycline for 48 hrs and subsequently fixed in ice-cold methanol generated and expression of αvβ5 integrin and scFv was assesed by immunofluorescence using anti-αvβ5 integrin clone P1F6, anti-c-myc 9E10 antibody and Texas re or Oregron green labelled secondary antibodies.

FIG. 28 shows expression of αvβ5 integrin and scFv in NIH:OVCAR-3 stable transfectant expressing the ER-VK-8-4.5 anti-CA125 scFv when induced or not with doxycycline. Cells were grown in the absence or presence of doxycycline for 48 hrs and subsequently fixed in ice-cold methanol generated and expression of αvβ5 integrin and scFv was assesed by immunofluorescence using anti-αvβ5 integrin clone P1F6, anti-c-myc 9E10 antibody and Texas re or Oregron green labelled secondary antibodies.

FIG. 29 shows alignment of deduced amino acid sequence for anti-CA125 scFvs VK-8-1.9 and OC125-3.11 as well as control scFv VK-8-4.5 Nucleotidic sequences encoding the anti-CA125 scFvs and their control were determined from pCANTAB5E/scFv constructs using the scFv specific primers S1 and S6 from the pCANTAB5 sequencing primer set (Amersham Pharmacia Biotech, Piscataway, N.J.). Sequences were determined using the LI-COR automatic sequencing system (Bio S&T Inc., Lachine, QUE). Amino acid sequence was deduced from the nucleotidic sequences and aligned using the Alibee multiple alignment software available at www.genebee.msu.su/services/malign reduced.html. The area in boxes represent consensus sequences of frameworks 1-4 of heavy and light chain, asterisks correspond to differences between VK-8-1.9 and OC125-3.11 anti-CA125 scFvs whereas arrows identify differences between VK-8-1.9 and VK-8-4.5 scFvs.

FIG. 30 shows SEQ ID NO: 1.

The 5′ to 3′ coding sequence of the head-to-tail linked V_{H}-linker-V_{L} portion of the single-chain antibody VK-8-1.9. The nucleotide sequence of the linker is underlined.

FIG. 31 shows SEQ ID NO: 2.

The 5′ to 3′ coding sequence of the head-to-tail linked V_{H}-linker-V_{L} portion of the single-chain antibody OC125-3.11. The nucleotide sequence of the linker is underlined.

FIG. 32 shows SEQ ID NO: 3.

The 5′ to 3′ coding sequence of the V_{H} portion of the single-chain antibody VK-8-1.9.

FIG. 33 shows SEQ ID NO: 4.

The 5′ to 3′ coding sequence of the V_{L} portion of the single-chain antibody VK-8-1.9.

FIG. 34 shows SEQ ID NO: 5.

The 5′ to 3′ coding sequence of the V_{H} portion of the single-chain antibody OC125-3.11.

FIG. 35 shows SEQ ID NO: 6.

The 5′ to 3′ coding sequence of the V_{L} portion of the single-chain antibody OC125-3.11.

While the invention will be described in conjunction with example embodiments, it will be understood that it is not intended to limit the scope of the invention to such embodiments. On the contrary, it is intended to cover all alternatives, modifications and equivalents as may be included as defined by the appended claims.

### DESCRIPTION

The present invention is directed to modulators of CA 125 tumor antigen, recombinant nucleic acids, vectors, host cells, pharmaceutical compositions, and methhods of use of the foregeoing for negatively modulating CA 125 tumor antigen in mammalian cell in order to prevent and treat a CA 125 tumor antigen associated disease in a mammal.

Modulators of the Present Invention

The present invention is directed to modulators capable of negatively modulating of CA 125 tumor antigen in a mammalian cell. More specifically, the modulators of the invention negatively modulate the function and or expression of CA 125 tumor antigen. Negative modulation is to be understood herein therefore as a significant decrease and preferably the abolition of the function and or expression of CA 125 tumor antigen. The present inventors show herein that CA 125 tumor antigen is responsible for the promotion of CA 125 associated diseases in mammals. More particularly, the inventors have discovered that CA 125 tumor antigen function and CA 125 tumor antigen expression both constitute novel therapeutic targets for combatting these diseases. The modulators of the present invention are therefore aimed at these novel therapeutic targets which are CA 125 tumor antigen expression and CA 125 tumor antigen function.

The CA125 tumor antigen is a protein associated with the majority of human epithelial ovarian cancers, the most common form of the disease. It is also known to be overexpressed in other diseases such as endometriosis, cervical cancer, cancer of the uterus, fallopian tube cancer, cancer of the endometrium, prostate cancer, lung cancer, etc. The foregoing diseases are therefore non exclusive examples of what is meant by the expression “CA 125 tumor antigen associated diseases”.

As used herein, the term “mammal” refers to any mammal that is susceptible to a CA 125 tumor antigen associated disease as defined herein. Among the mammals which are known to be potentially affected, are humans.

More specifically, the present invention concerns a modulator capable of negatively modulating CA 125 tumor antigen function and or expression in a mammalian cell. Such a modulator may, for instance, negatively modulate the cell surface expression of CA 125 tumor antigen. One way of achieving this downregulation of CA 125 tumor antigen cell surface expression is through the sequestration of newly synthesized CA 125 tumor antigens or peptidic fragments thereof within cellular organelle(s) such as the endoplasmic reticulum, the trans-golgi, the golgi, the mitochondrion, the cytoplasm. The sequestration may also alternatively be achieved within any other cellular compartment.

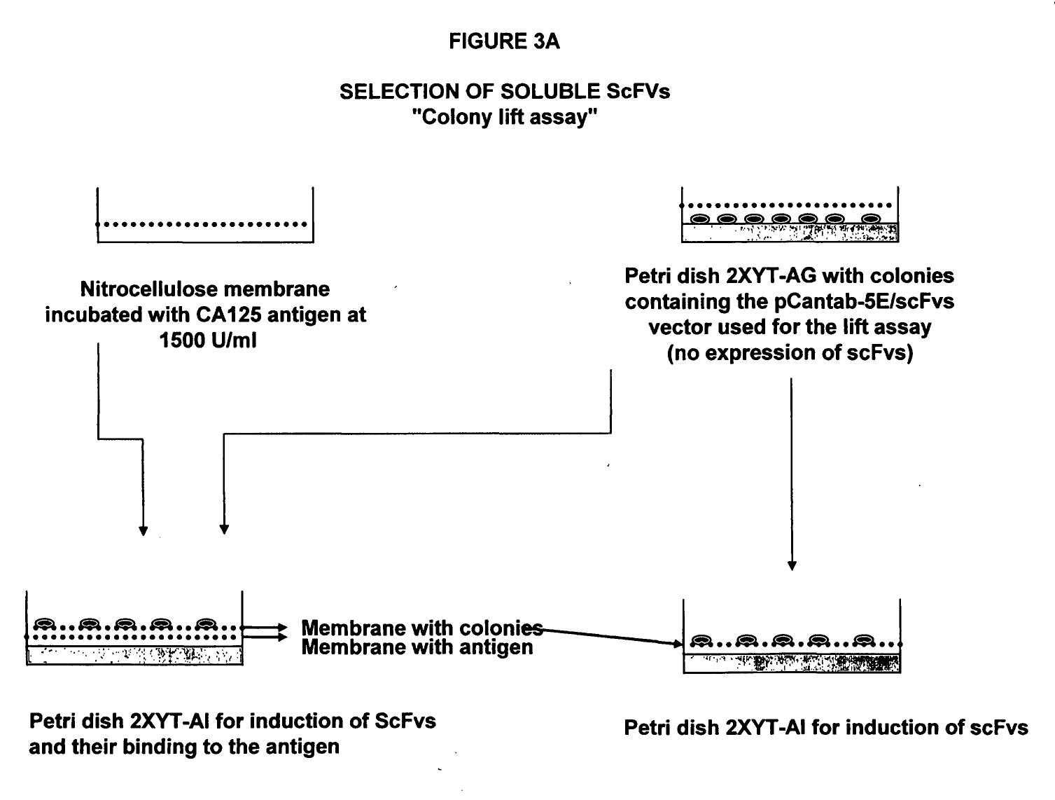

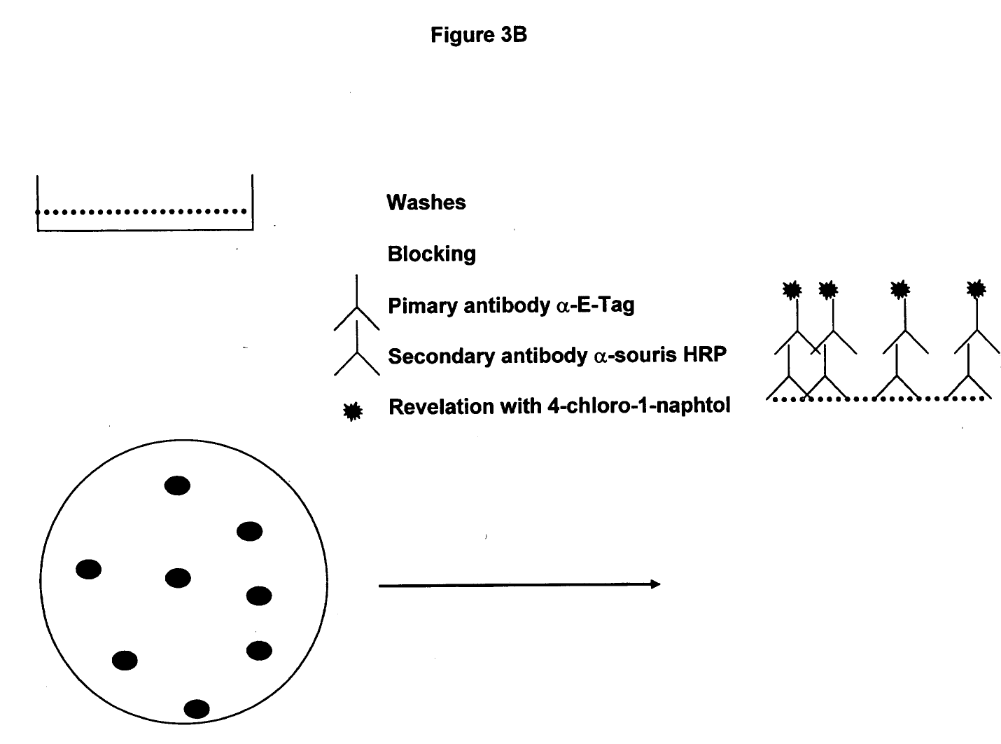

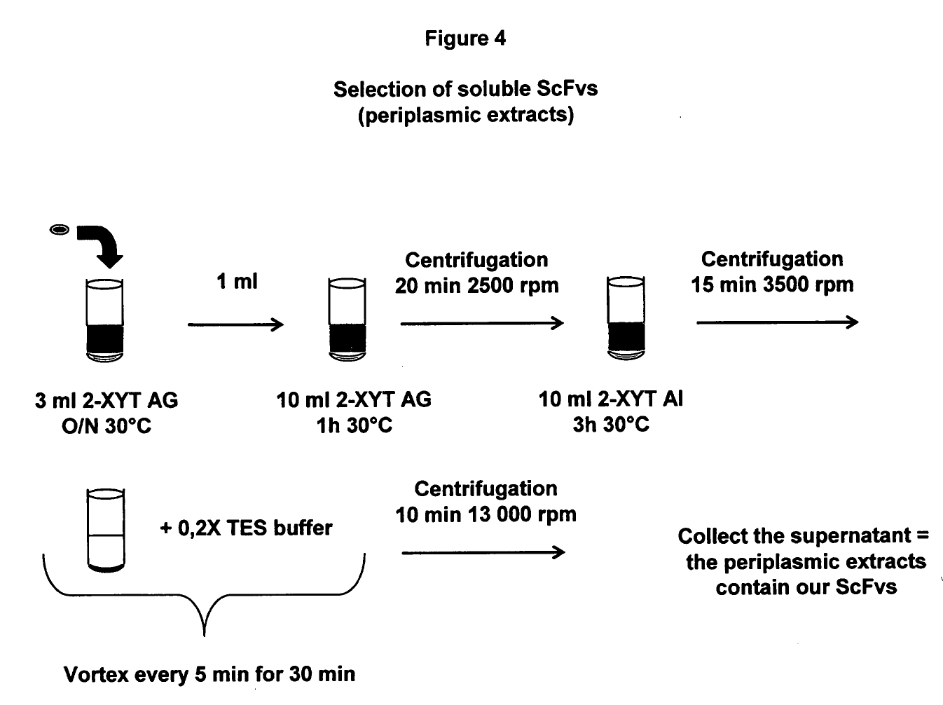

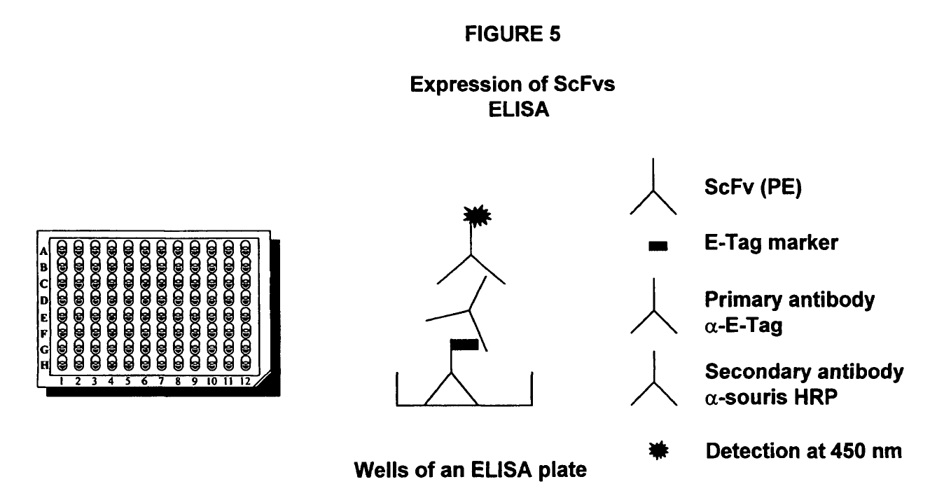

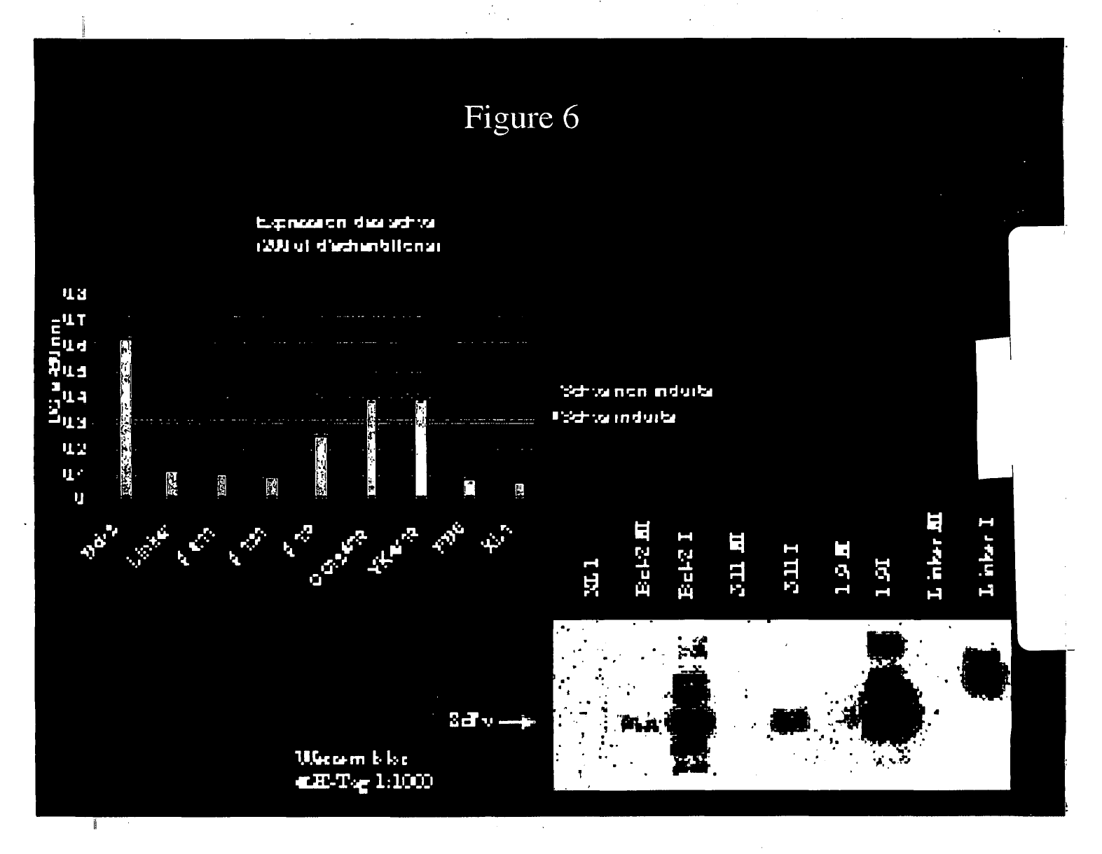

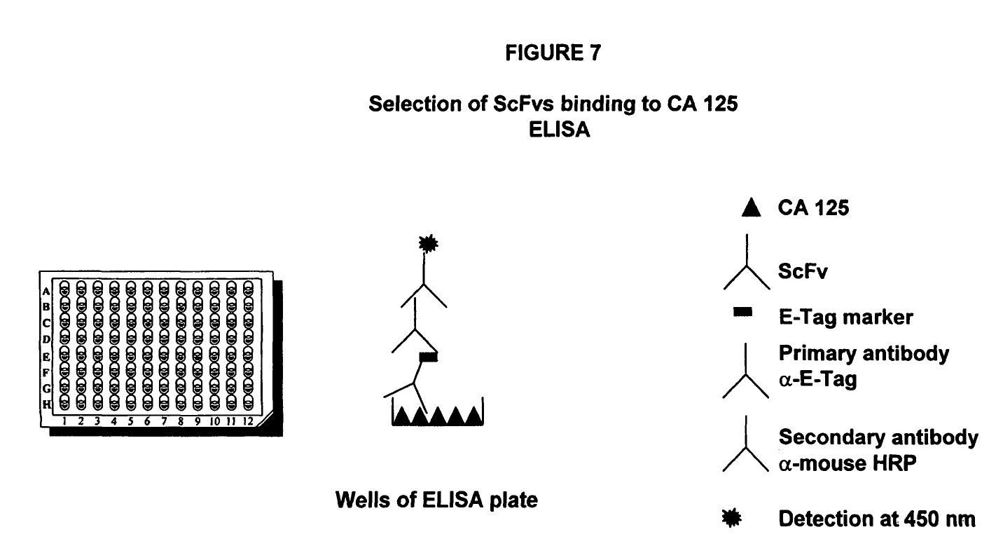

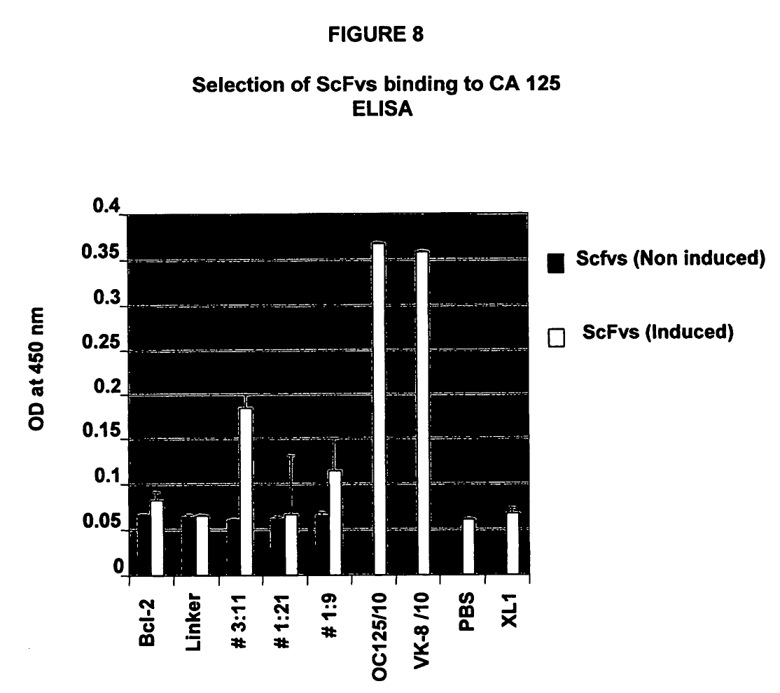

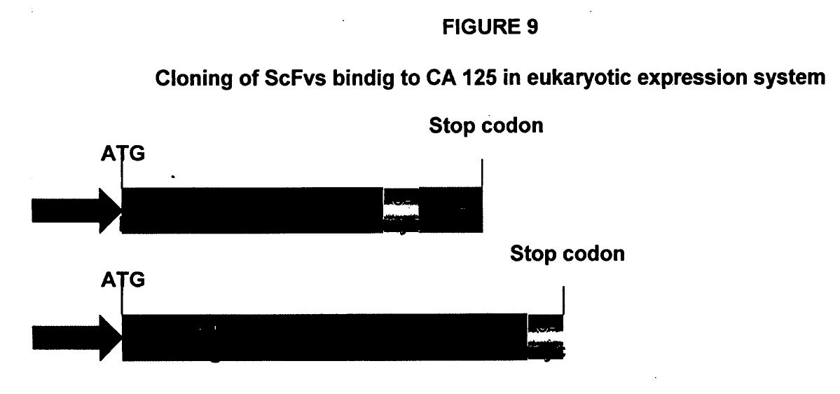

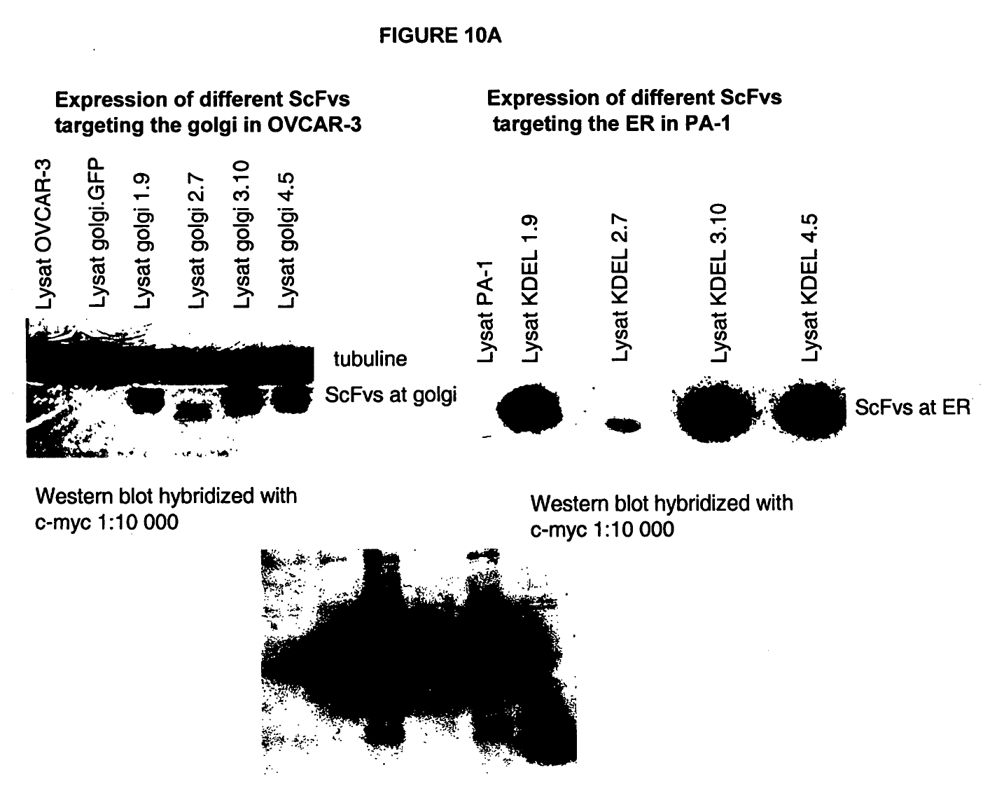

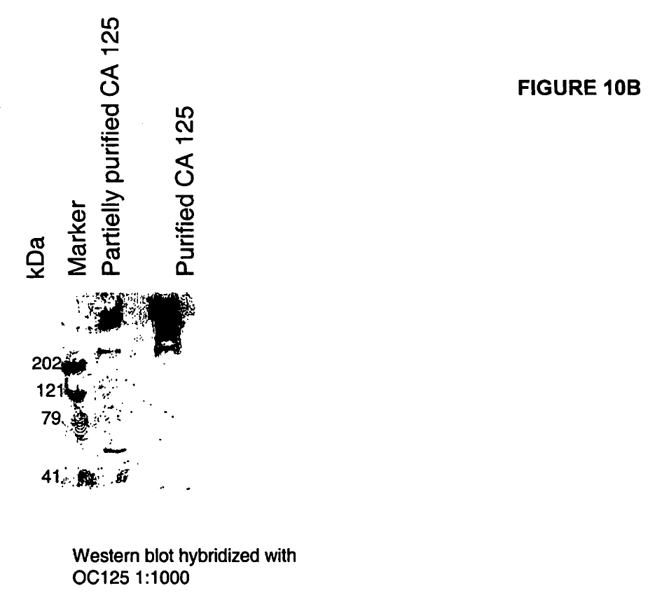

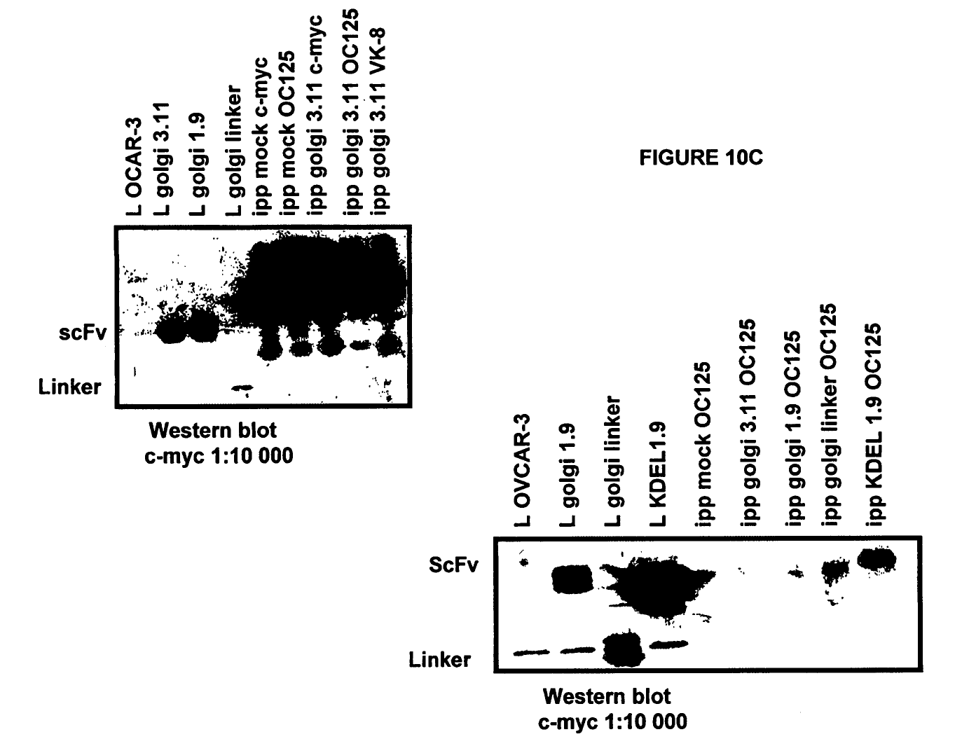

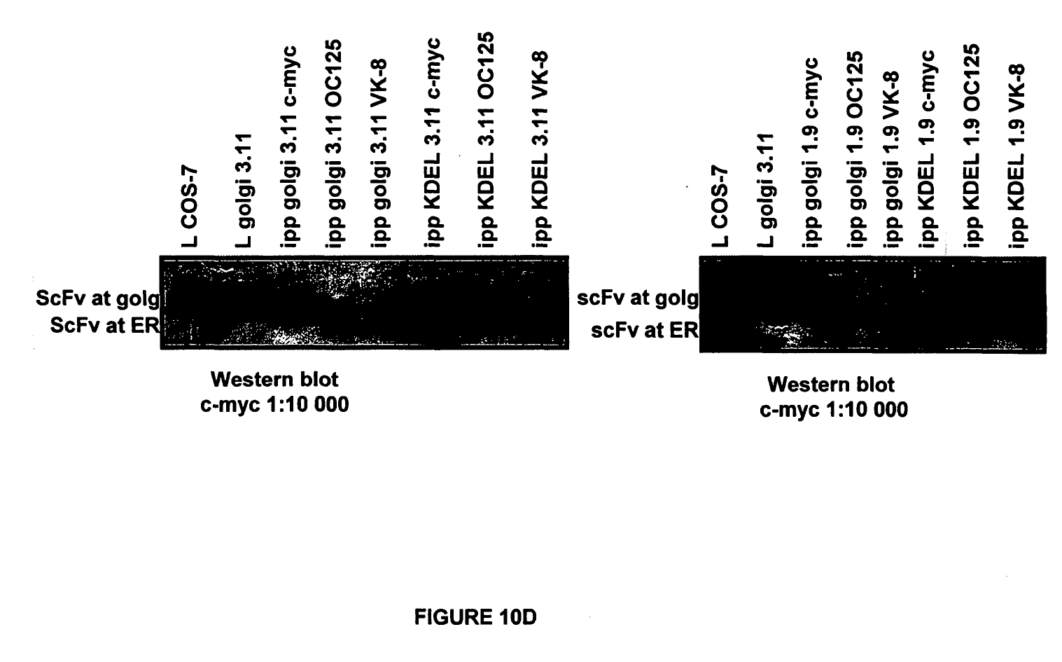

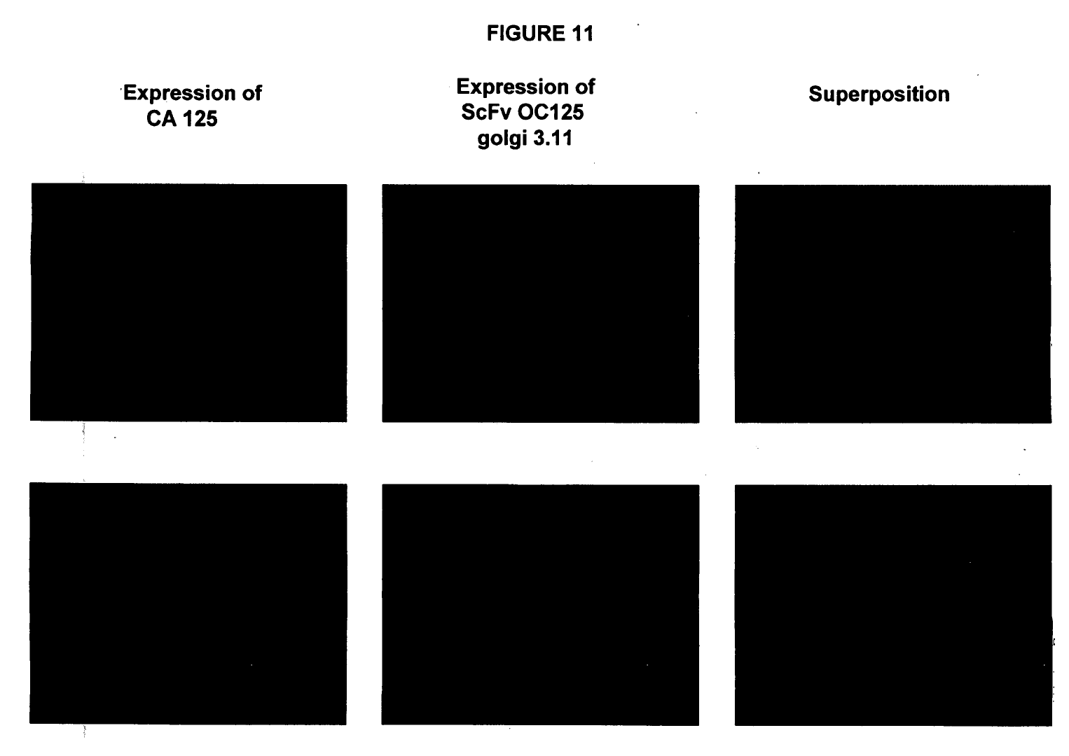

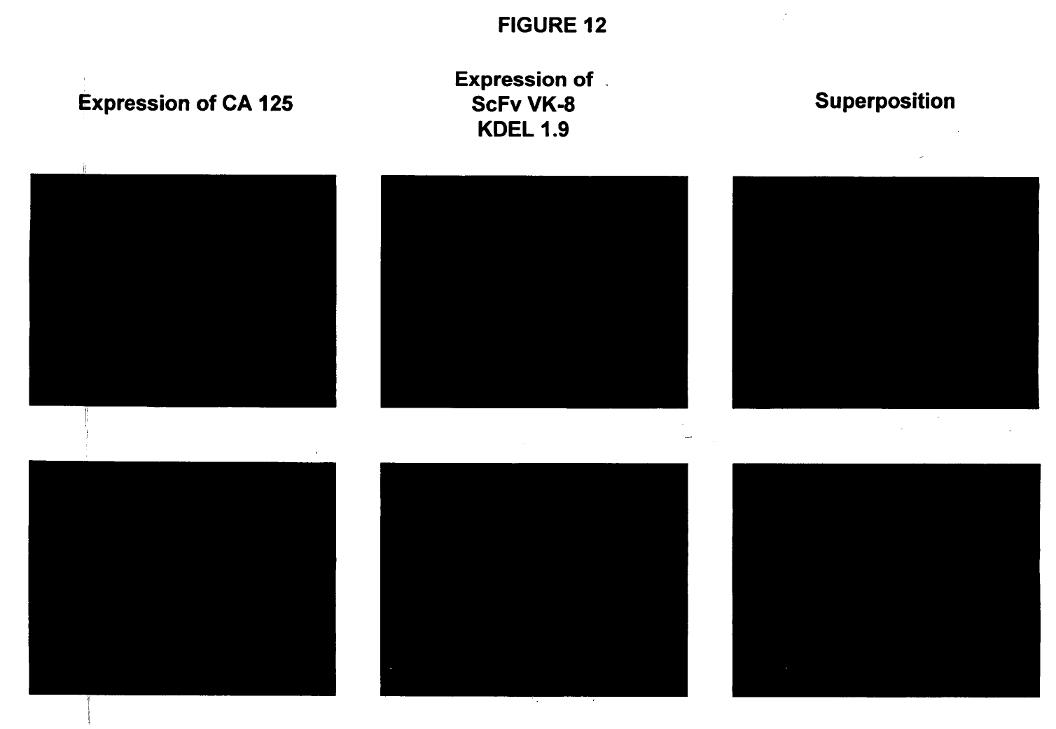

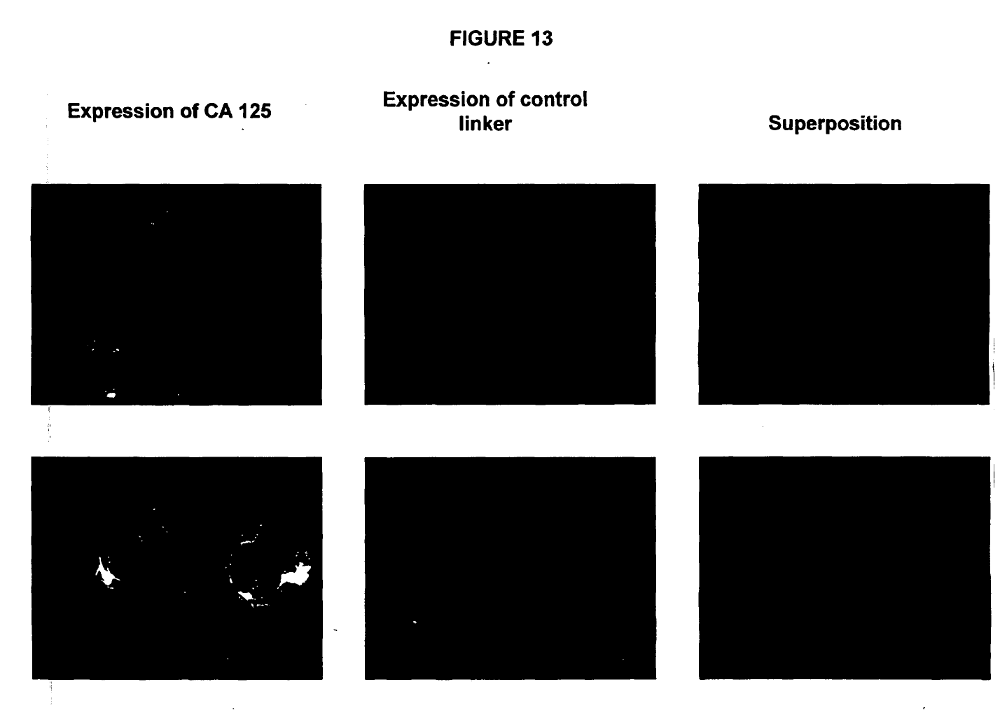

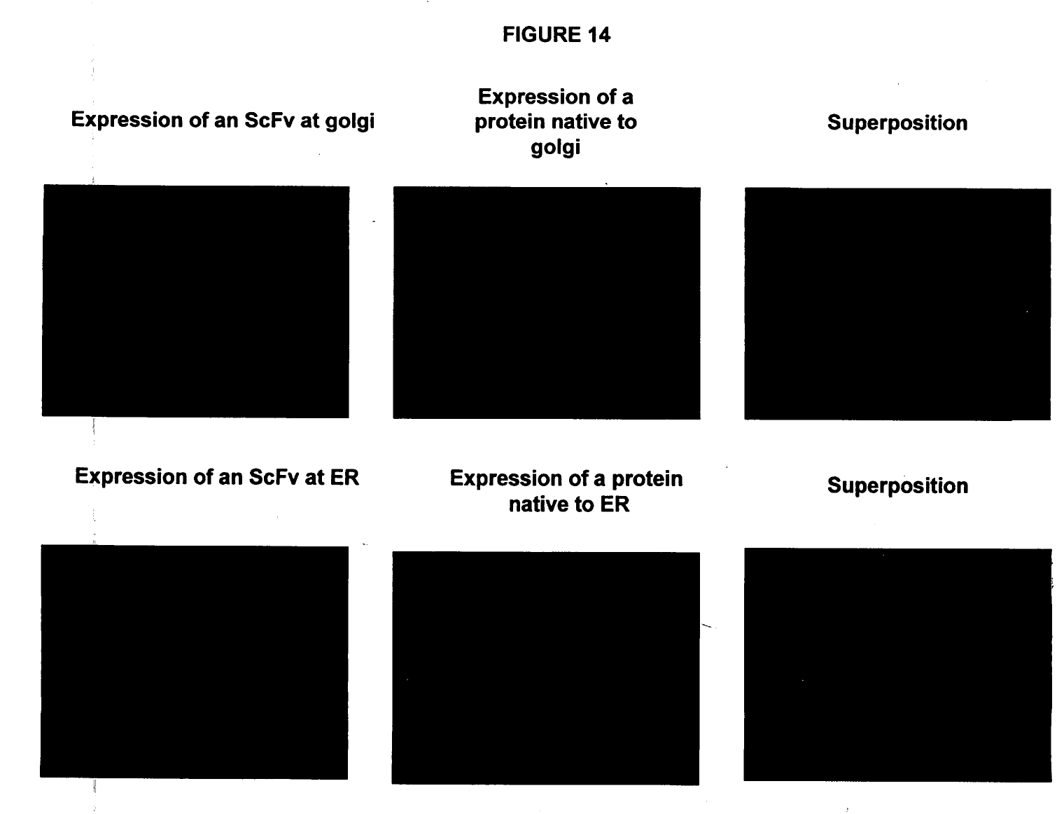

A prefered modulator contemplated by the present invention is a single-chain antibody that specifically binds to CA 125 tumor antigen or a peptidic fragment thereof. Such a single-chain antibody is preferably derived from the OC 125 monoclonal antibody or the VK-8 monoclonal antibody as are the single-chain antibodies generated by the inventors and designated hereinbelow OC 125-3.11 and VK-8-1.9. These particular single-chain antibodies contain fragments coded by SEQ ID Nos 2 and 1 respectively (FIGS. 31 and 30). However, the invention also concerns any single-chain antibody comprising a fragment coded by any one or more of the sequences of SEQ. ID NOS 1 to 6 (FIGS. 30 to 35) and which specifically binds to CA 125 tumor antigen or a polypeptidic fragment thereof and, as discussed herein, negatively modulates CA 125 tumor antigen function and or expression. Such an antibody preferably results from the combination, for instance, of the variable heavy sequence derived from the OC 125 monoclonal antibody (FIG. 5) linked to the variable light sequence derived from the VK-8 monoclonal antibody (FIG. 4). Or, preferably it results from the permutaion of the variable heavy sequence derived from the VK-8 monoclonal antibody (FIG. 3) linked to the variable light sequence derived from the OC 125 monoclonal antibody (FIG. 6). Other such permutations of SEQ ID Nos 1 to 6 are also withhin the scope of the present invention.

The present inventors developed the two above mentioned anti-CA125 single-chain antibodies (scFvs), OC 125-3.11 and VK-8-1.9, and show that they act as CA125-specific negative modulators of CA 125 tumor antigen function and expression as further described hereinbelow. When expressed intracellularly and retained to the ER (endoplasmic reticulum) or Golgi, the anti-CA125 scFvs entrap CA125 within the secretion pathway and therefore prevent its proper cell surface localization in the mammalian cells which results in an increased cell proliferation and sensitivity to chemotherapeutic drugs such as cisplatin, reduced cell adhesion and migration and prevents tumor growth in nude mice. The inventors show a functional link between CA125 tumor antigen and cell proliferation, sensitivity to drugs such as cisplatin, cell adhesion and cell migration and tumorigenicity.

Alternatively, the negative modulators of the present invention are to be understood as comprising any negative modulator of CA 125 tumor antigen function and or expression. As people skilled in the art will know, such negative modulators may act at different levels: such as the transciptional, post-transcriptional, translational or post-translational levels. Such negative modulators may be any type of ligand that specifically bind any CA 125 tumor antigen precursor (such as a CA 125 mRNA), or any CA 125 tumor antigen, or fragments of the foregoing. Dominant negative molecules are another example of a negative modulator which allows to achieve the negative modulation of CA 125 tumor antigen function and or expression. A negative modulator of the present invention may therefore be any molecule which specifically inhibits, blocks, neutralizes, knocks-out, or downregulates CA 125 tumor antigen function and or expression in a mammalian cell. Such a negative modulator may also act upon CA 125 tumor antigen function or expression from the outside of the cell, for instance, through the extracellular portion of CA 125 tumor antigen.

Recombinant Nucleic Acids

The present invention is also directed to any recombinant nucleic acid comprising at least one nucleic acid sequence selected from the group consisting of SEQ ID NOS 1 to 6 (FIGS. 30 to 35 respectively).

The expression “nucleic acid sequence”, “nucleotide sequence”, “nucleic acid”, “polynucleotide”, “polynucleotide sequence” are terms which are employed interchangeably in the present application and which are meant to designate a precise chain of nucleotides, modified or not,allowing the defintion of a fragment or region of a polynucleotide, comprising or not non natural nucleotides, and that may correspond to double-stranded DNA or a single-stranded DNA. This also includes DNA molecules, RNA molecules, cDNA, artificial sequences and all fragments thereof. Any polynucleotide which has been chemically, enzymatically, or metabolically modified but which still retains the properties of the original polynucleotide, e.g. codes for a peptide fragment which specifically binds to CA 125 tumor antigen in mammalian cells, is included within the meaning of the present invention.

The present invention does not concern nucleotide sequences in their natural chromosome environment, e.g. in the natural state. Rather it concerns purified or isolated sequences, e.g. sequences that have been directly or indirectly obtained, through a process such as cloning, amplification and or chemical synthesis, their environment having therefore been at least partially modified. It is also understood that a polynucleotide which is introduced in an organism by transformation, genetic engineering, or any other recombinant method is “isolated” or “recombinant” even though it is present inside the said organism.

The sequences of the present invention preferably possess a DNA sequence presenting a percentage of identity of 70% or more with one of the sequences of FIGS. 30 to 35. The expression “percentage of identity” is meant to indicate a degree of identity between two nucleic acid sequences along the sequences in their entirety. If the particular sequences are of different lengths, the percentage of identity is expressed relatively to the total length of the shortest sequence of the two. In order to calculate the percentage of identity, both sequences are superposed in such a way to maximize the number of identical bases allowing for intervals, the number identical bases is is then divided by the total number of bases of the shortest sequence.

As used herein, the term “polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may contain many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of cysteine, formation of pyroglutamate, formylation, gamma-carboxylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation, selenoylation, sulfation and transfer-RNA mediated addition of amino acids to proteins, such as arginylation, and ubiquitination. See, for instance: PROTEINS—STRUCTURE AND MOLECULAR PROPERTIES, 2nd Ed., T. E. Creighton, W.H. Freeman and Company, New York (1993); Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York (1983); Seifter et al., Meth. Enzymol. 182:626-646 (1990); and Rattan et al., Protein Synthesis: Posttranslational Modifications and Aging, Ann. N.Y. Acad. Sci. 663: 48-62(1992). Polypeptides may be branched or cyclic, with or without branching. Cyclic, branched and branched circular polypeptides may result from post-translational natural processes and may be made by entirely synthetic methods, as well.

Vectors and Host Cells

In a third embodiment, the invention is further directed to cloning or expression vector comprising a polynucleotide sequence as defined above, and more particularly directed to a cloning or expression vector which is capable of directing expression of the polypeptide encoded by the polynucleotide sequences of the present invention in a vector-containing cell or host cell.

As used herein, the term “vector” refers to a polynucleotide construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, or a “viral vector” which is designed to result in the production of a recombinant virus or virus-like particle, or “shuttle vectors”, which comprise the attributes of more than one type of vector.

A number of vectors suitable for stable transfection of cells and bacteria are available to the public (e.g. plasmids, adenoviruses, baculoviruses, yeast baculoviruses, plant viruses, adeno-associated viruses, retroviruses, Herpes Simplex Viruses, Alphaviruses, Lentiviruses), as are methods for constructing such cell lines. It will be understood that the present invention encompasses any type of vector comprising any of the polynucleotide molecule of the invention.

In a fourth embodiment, the invention is also directed to a host, such as a genetically modified cell, comprising a modulator of the present invention, and or a vector of the present invention, and or any of the polynucleotide sequences according to the invention and more preferably, a host capable of expressing the polypeptide encoded by this polynucleotide.

The host cell is any type of cell. Preferably, it is a mammalian cell, such as a cell from an estblished cell line or an isolated primary cell. Alternatively, it may be an insect cell, yeast cell (Saccharomyces cerevisiae, Ktuyveromyces lactis, Pichia pastoris), plant cell, microorganism, or a bacterium (such as E. coli).

Pharmaceutical Compositions

In a fifth embodiment, the present invention concerns any pharmaceutical composition comprising a pharmaceutically acceptable carrier and at least one element selected from the group consisting of a modulator as defined by the present invention, a recombinant nucleic acid as defined by the present invention, a vector as defined by the present invention, and a host cell as defined by the present invention.

Methods well-known in the art for making pharmaceutical compositions or formulations are found, for example, in “Remington's Pharmaceutical Sciences” (Gennaro A R ed., 20th edition, 2000: Williams & Wilkins PA, USA). Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Conventional pharmaceutical practice may be used to provide suitable formulations to administer the composition to patients. Administration may begin before the patient is symptomatic. Any appropriate route of administration may be employed, for example, administration may be parenteral, intravenous, intraarterial, subcutaneous, intramuscular, intracranial, intraorbital, ophthalmic, intraventricular, intracapsular, intraspinal, intracisternal, intraperitoneal, intranasal, aerosol, by suppositories, or oral administration. Therapeutic formulations may be in the form of liquid solutions or suspensions; for oral administration, formulations may be in the form of tablets or capsules; and for intranasal formulations, in the form of powders, nasal drops, or aerosols.

The amount of the elements in the composition of the present invention and amounts to be administered is a therapeutically effective amount. A therapeutically effective amount is that amount necessary for obtaining beneficial results without causing overly negative secondary effects in the host to which the composition is administered. The exact amount of each of the component elements in the composition and amount of the composition to be administered will vary according to factors such as the type of condition being treated, the other ingredients in the composition, the mode of administration, the age and weight of the individual, etc. If necessary, one may refer to the last edition of the Canadian Compendium of Pharmaceuticals & Specialties (CPS).

Uses and Methods

In a sixth embodiment, the present invention also concerns a use of at least one element selected from the group consisting of a modulator as defined by the present invention, a recombinant nucleic acid as defined by the present invention and a vector as defined by the present invention for negatively modulating a CA 125 tumor antigen in a mammalian cell.

In a seventh embodiment, the present invention concerns a use of at least one element selected from the group consisting of a modulator as defined by the present invention, a recombinant nucleic acid as defined by the present invention, a vector as defined by the present invention, a host cell as defined by the present invention and a pharmaceutical composition as defined by the present invention for preventing and treating a CA 125-tumor-antigen-associated disease in a mammal.

In an eighth embodiment, the present invention concerns a method of prevention or treatment of a CA 125-tumor-antigen-associated disease in a mammal comprising the step of administrating to that mammal at least one element selected from the group consisting of a modulator as defined by the present invention, a recombinant nucleic acid as defined by the present invention, a vector as defined by the present invention, and a host cell as defined by the present invention.

As used herein, the term “treating” refers to a process by which the symptoms of a CA 125 tumor antigen associated disease are alleviated or completely eliminated. As used herein, the term “preventing” refers to a process by which a CA 125 tumor antigen associated disease is obstructed or delayed.

In a ninth embodiment, the present invention concerns a method for negatively modulating a CA 125 tumor antigen in a mammalian cell comprising the step of introducing into that cell at least one element selected from the group consisting of a modulator as defined by the present invention, a recombinant nucleic acid as defined by the present invention, and a vector as defined by the present invention.

The present invention will be more readily understood by referring to the following examples. Thess examples are illustrative of the wide range of applicability of the present invention and are not intended to limit its scope. Modifications and variations can be made therein without departing from the spirit and scope of the invention. Although any methods and materials similar or equivalent to those described herein can be used in practice for the testing of the present invention, the preferred methods and materials are described.

### EXAMPLES

### Example I

### Construction and in vitro Validation of Modulators According to a Preferred Embodiment of the Present Invention, Namely, anti-CA125 scFvs

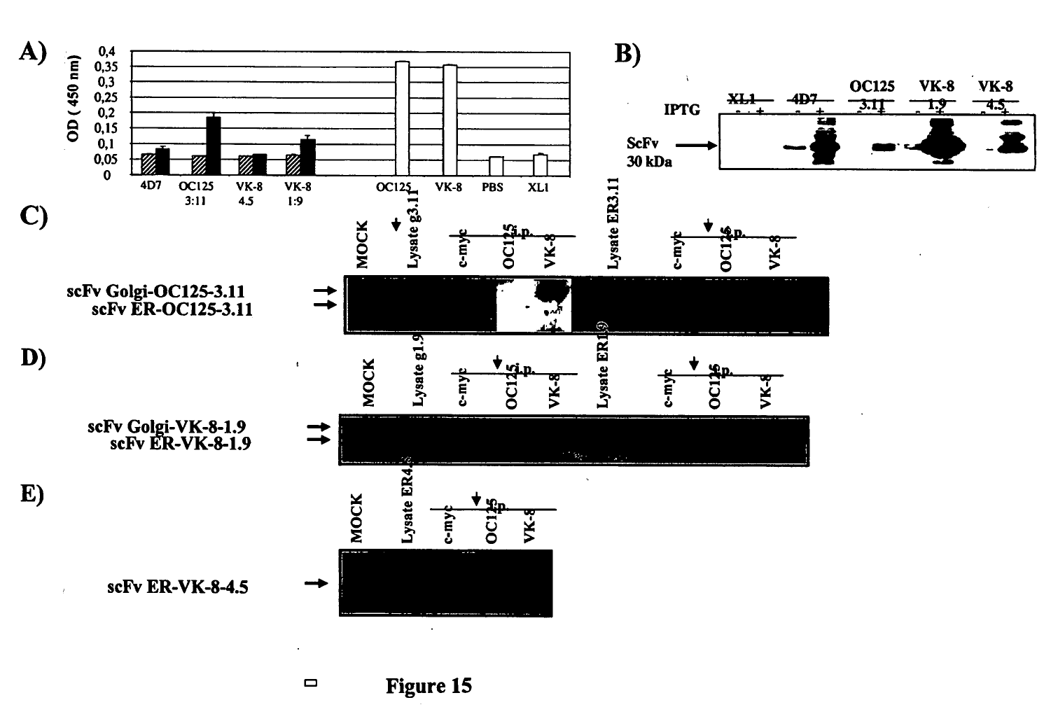

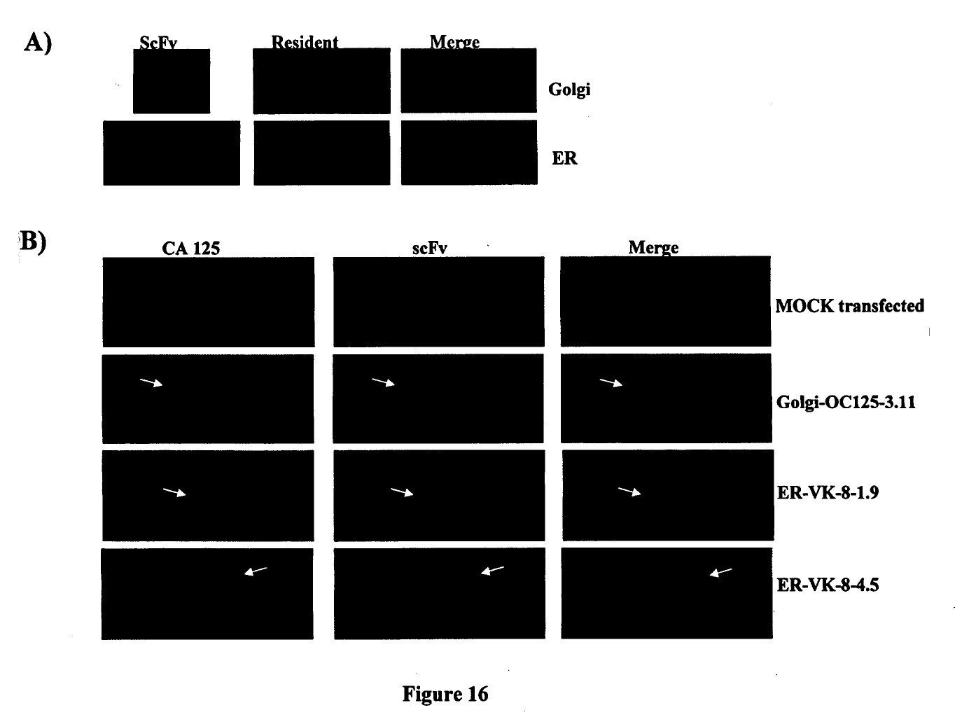

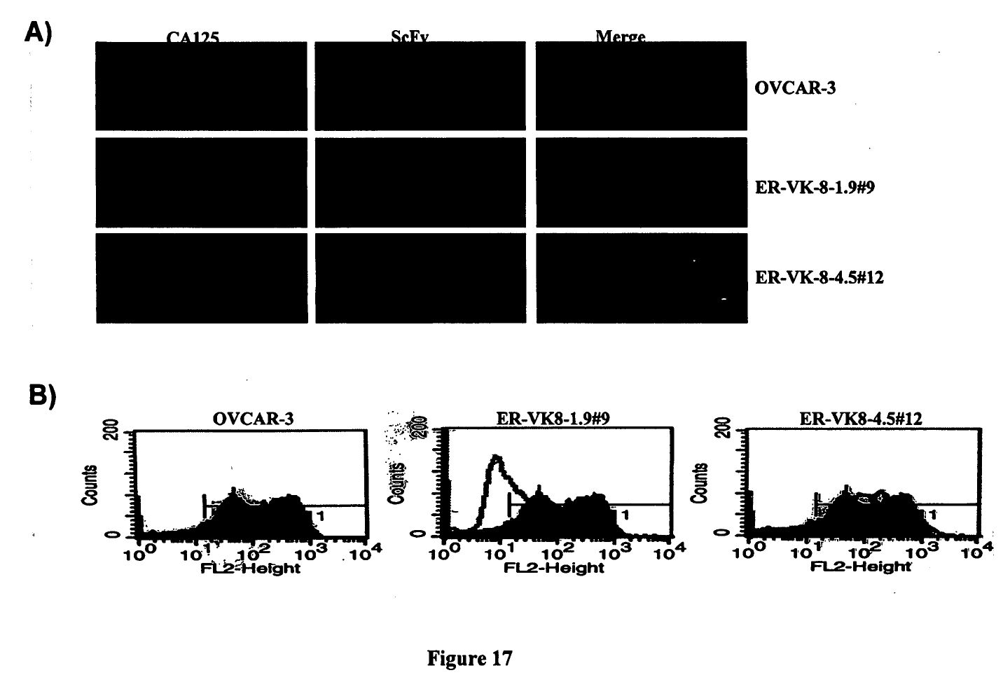

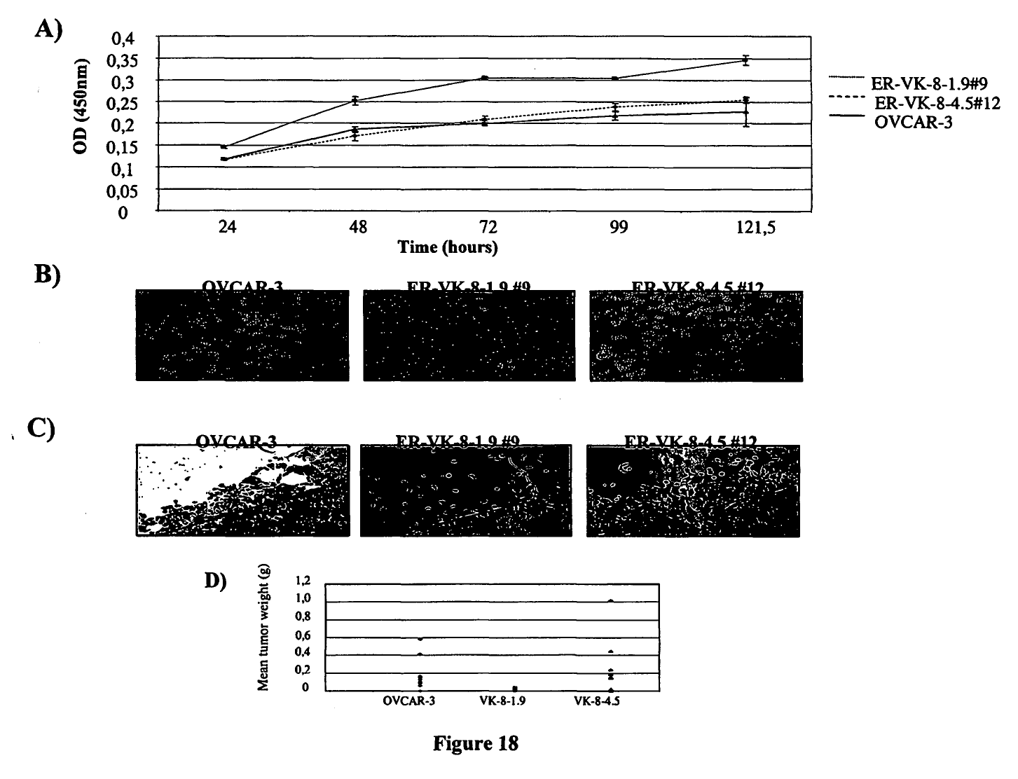

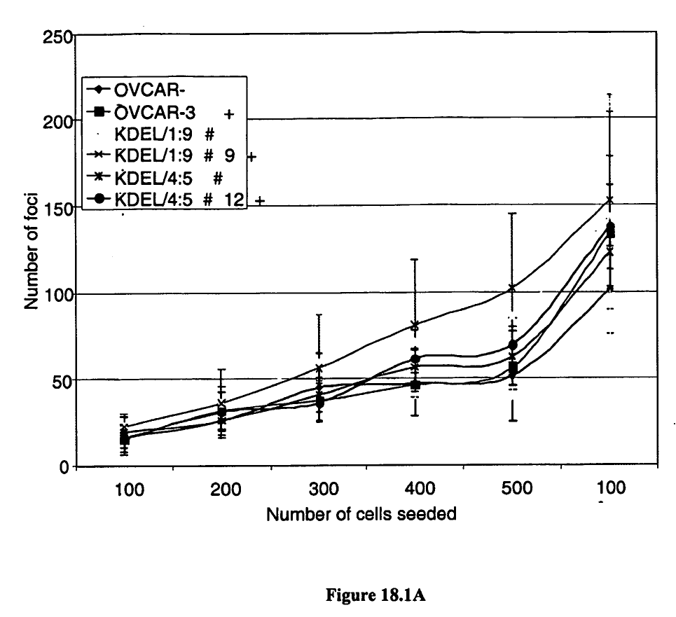

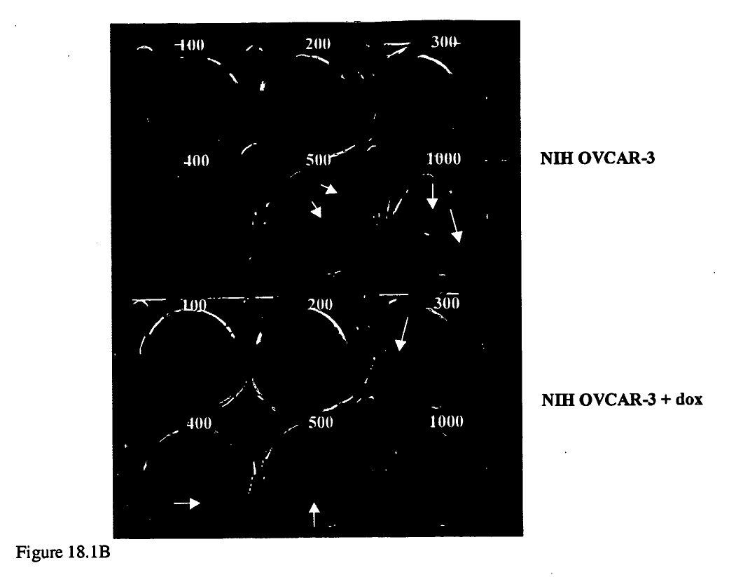

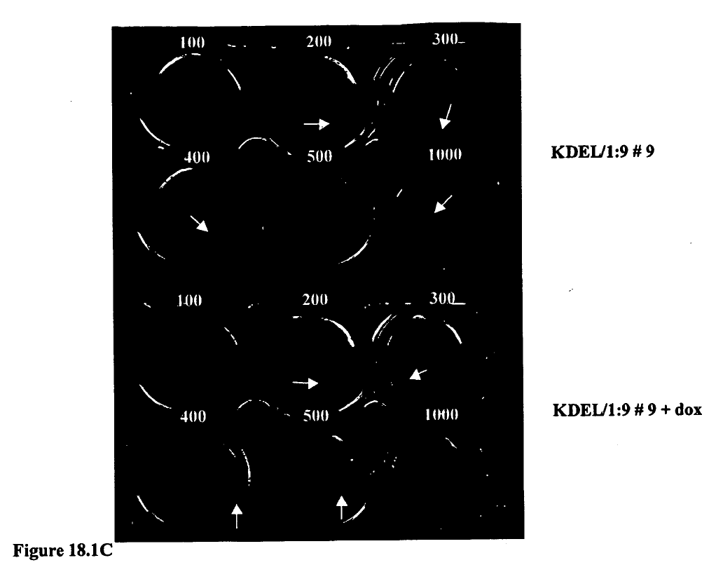

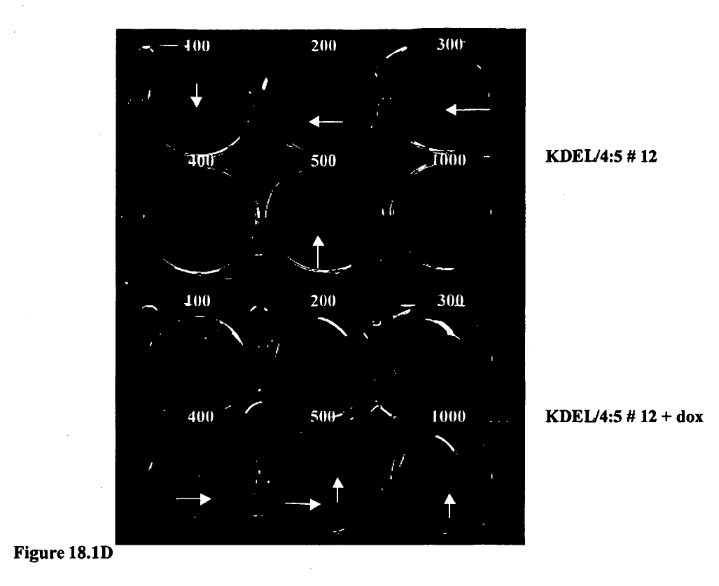

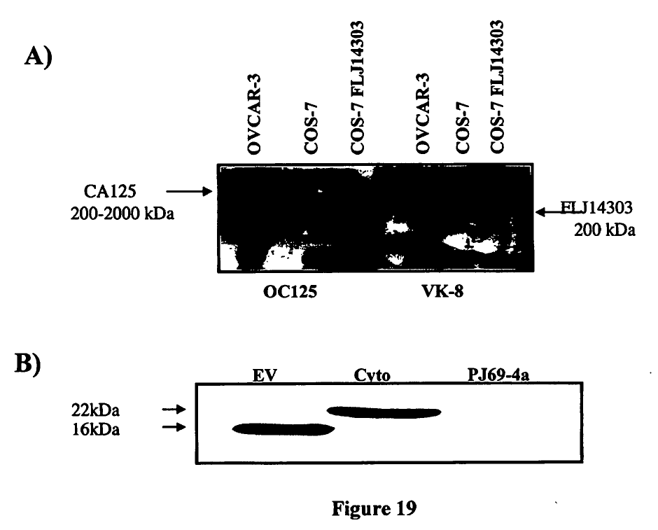

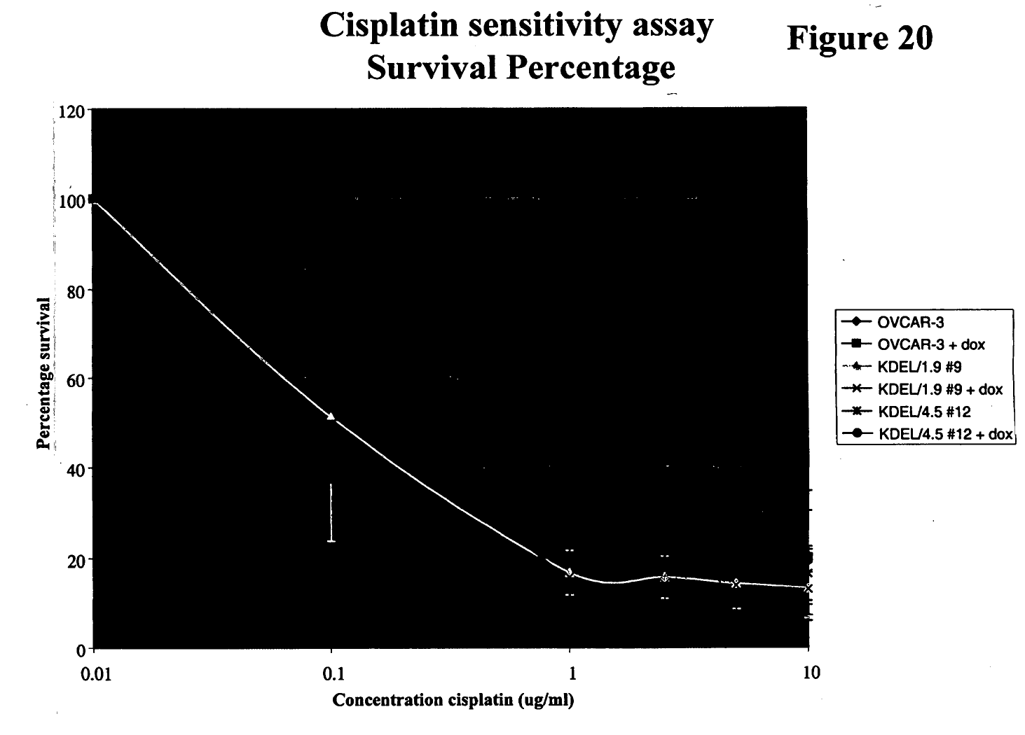

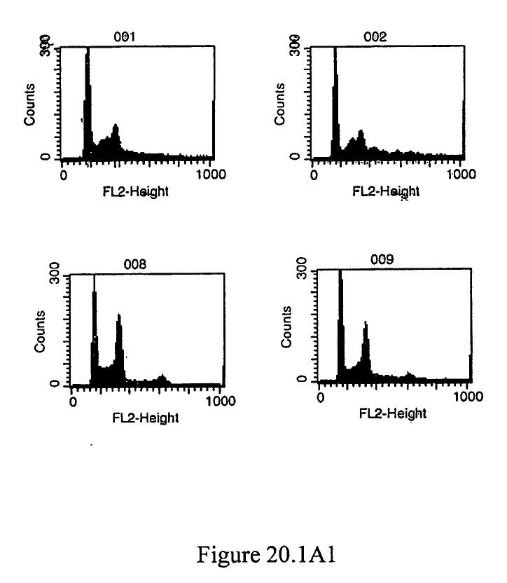

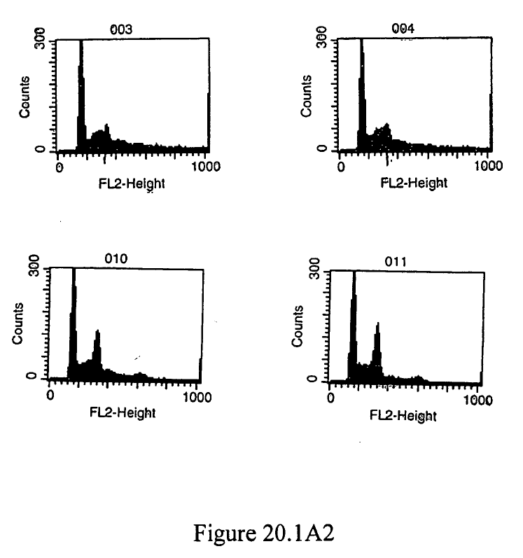

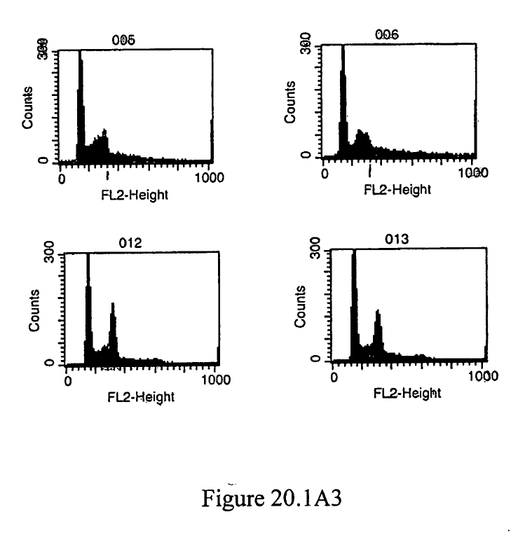

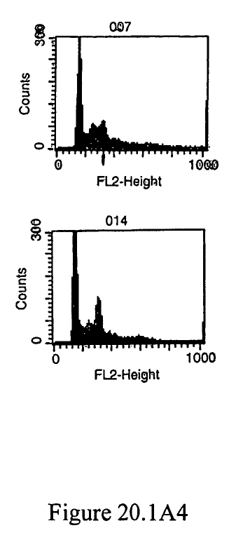

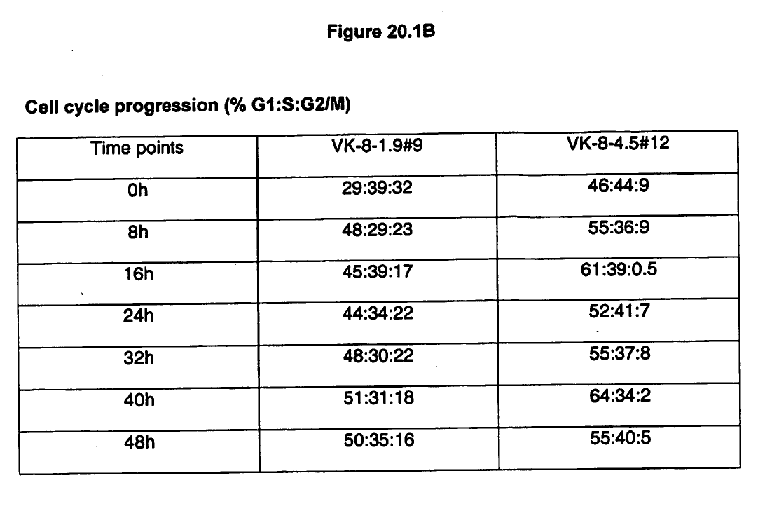

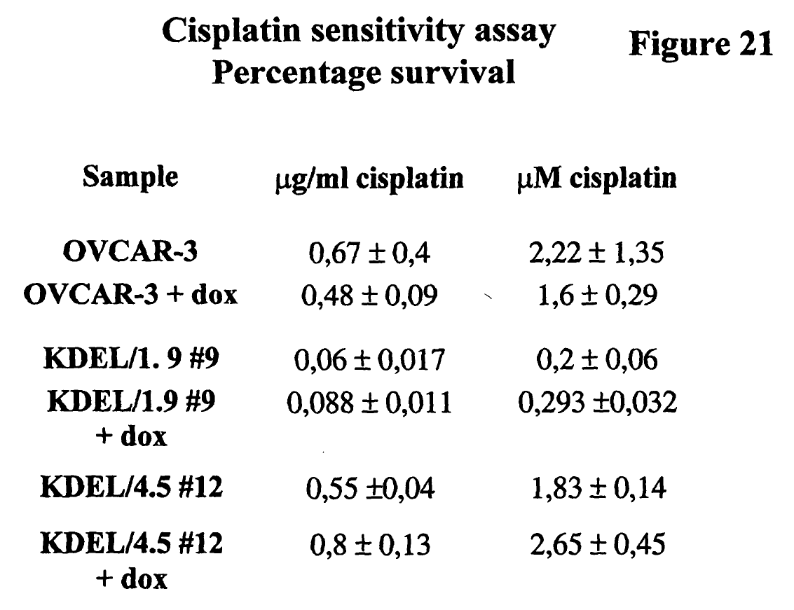

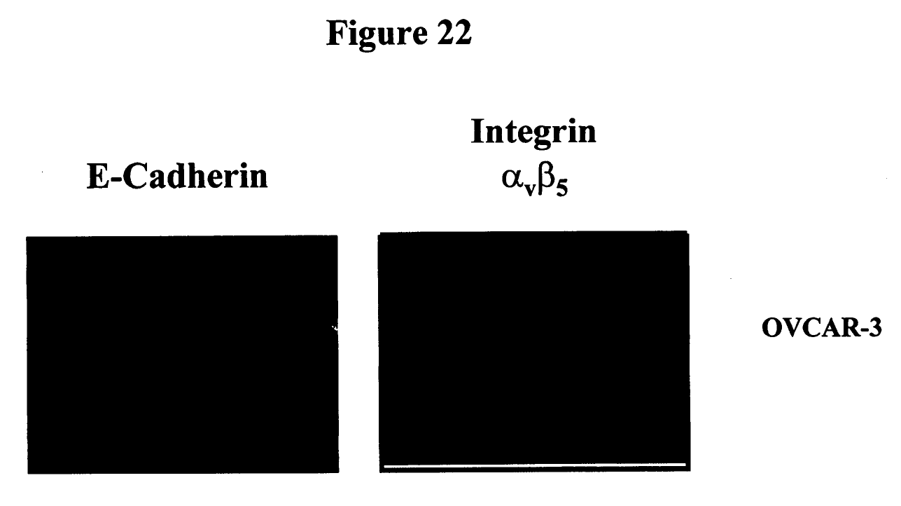

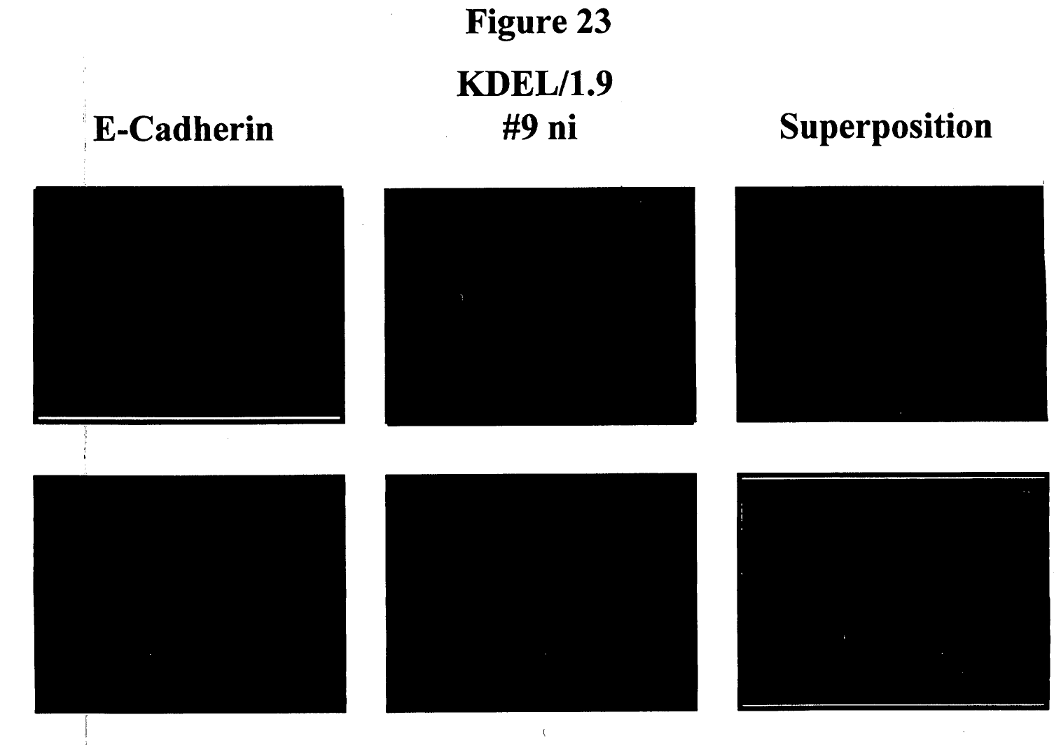

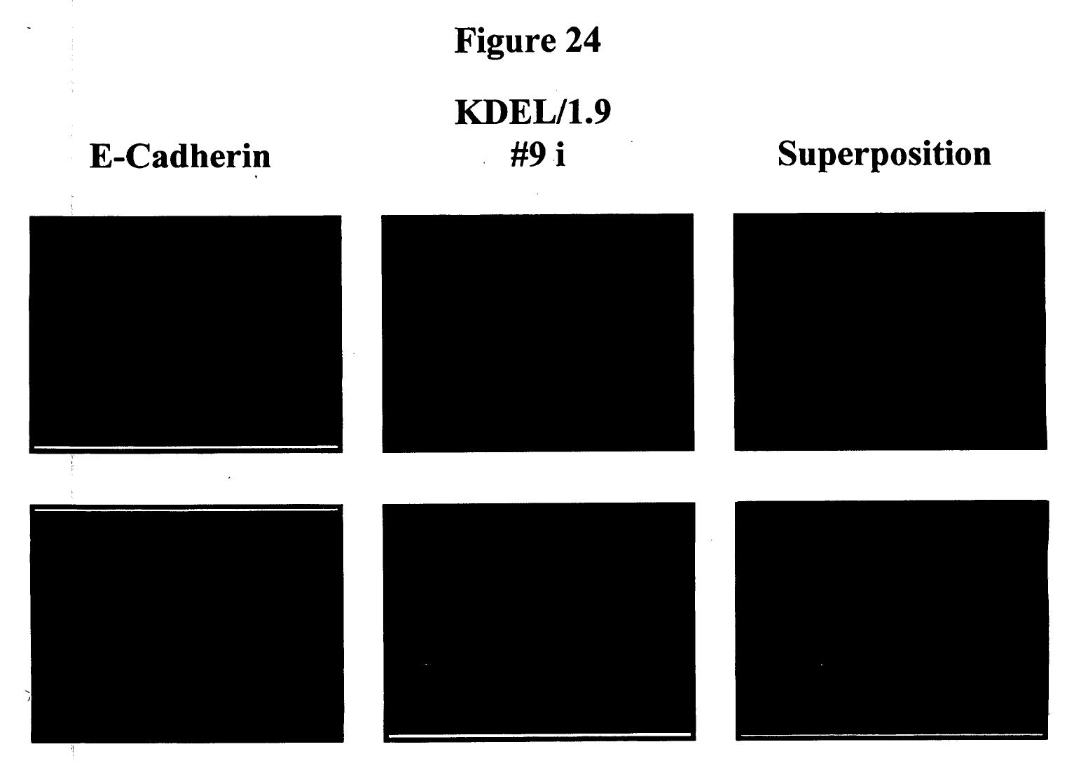

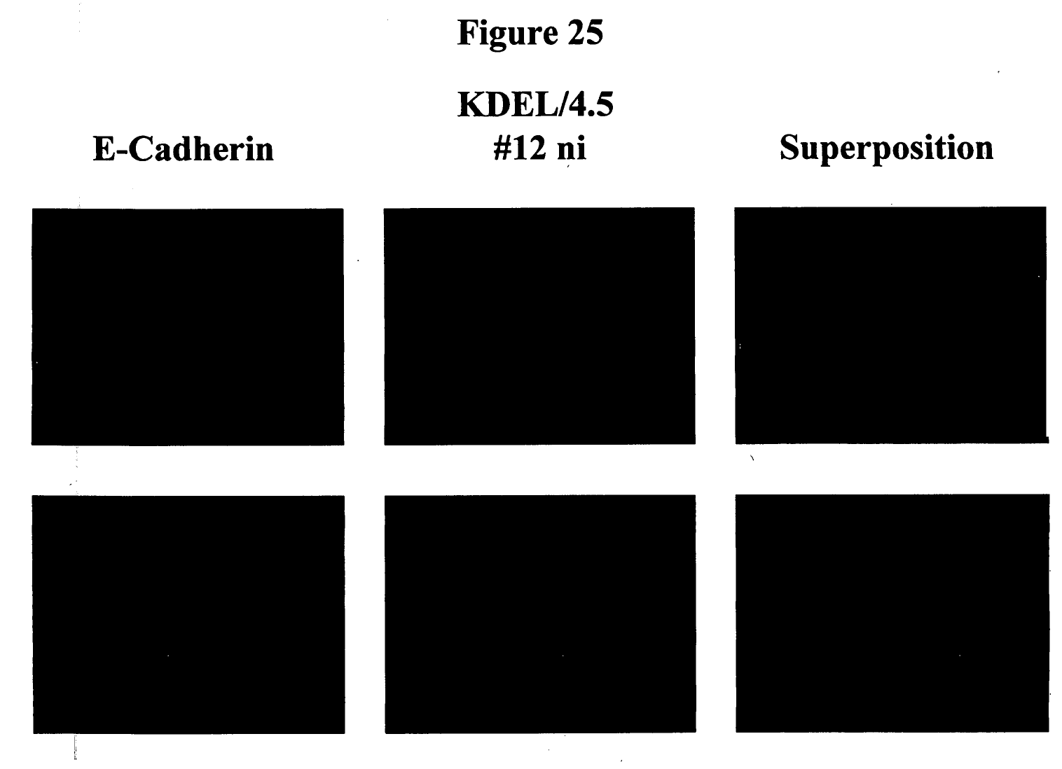

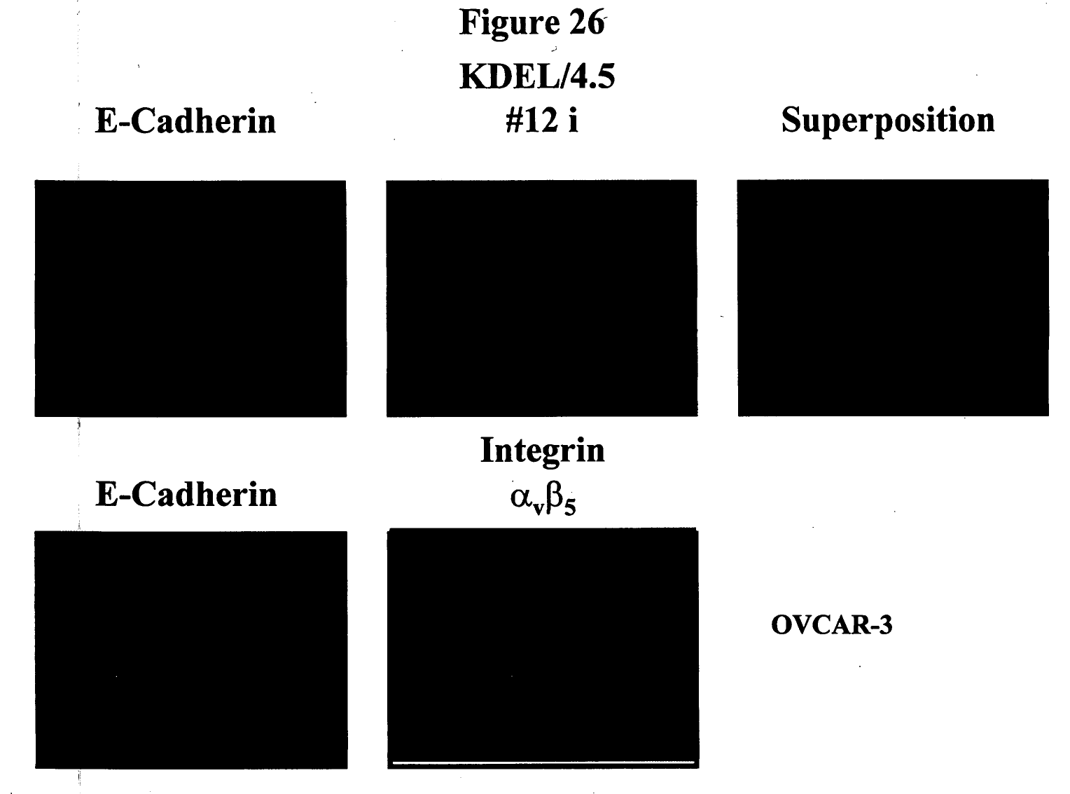

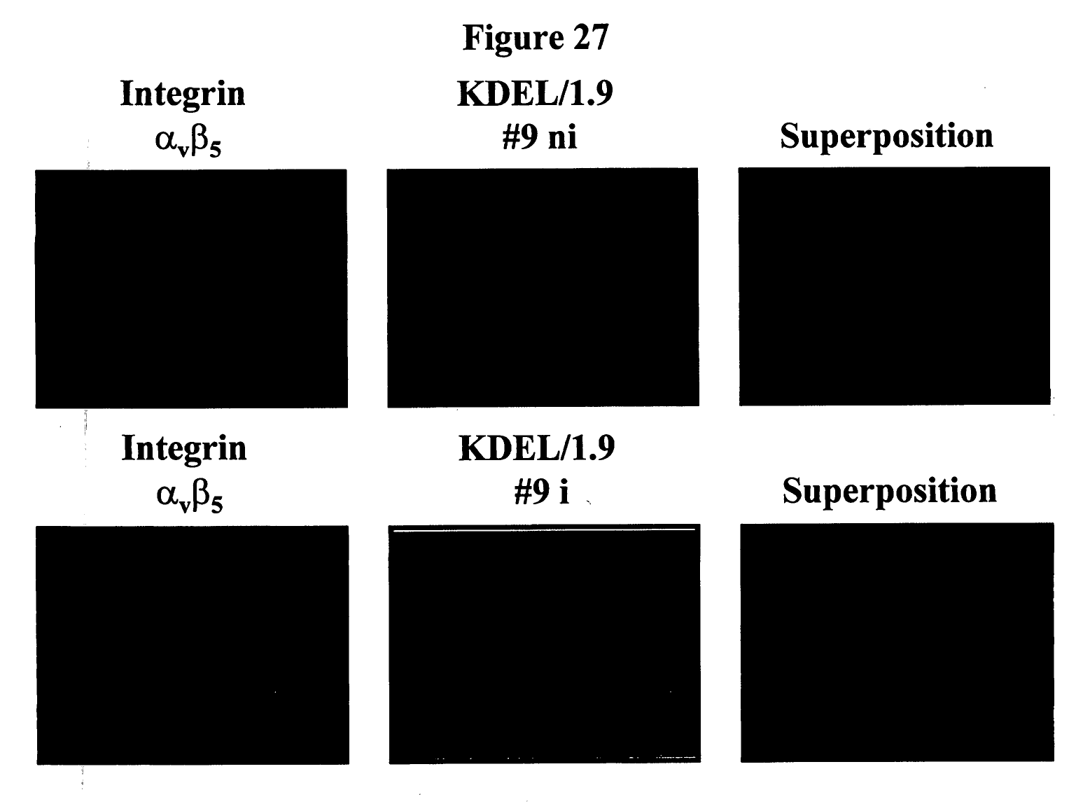

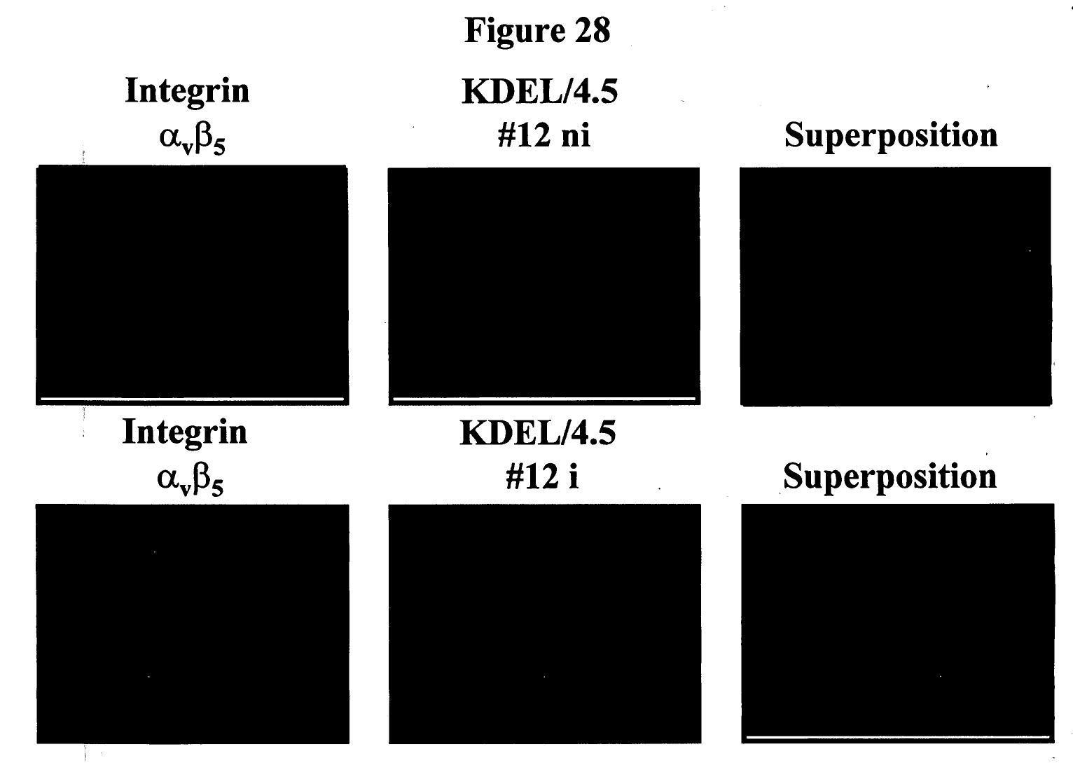

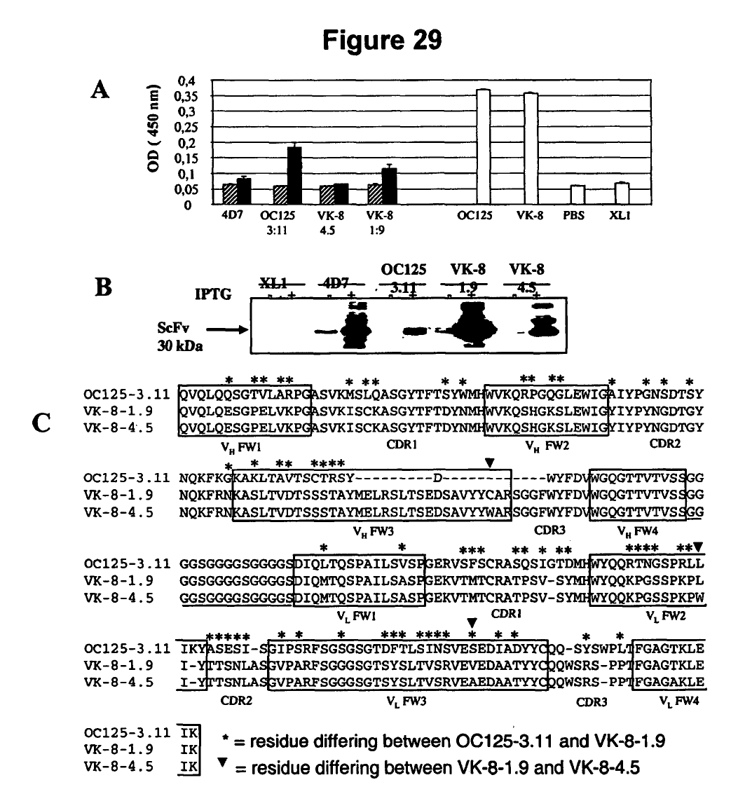

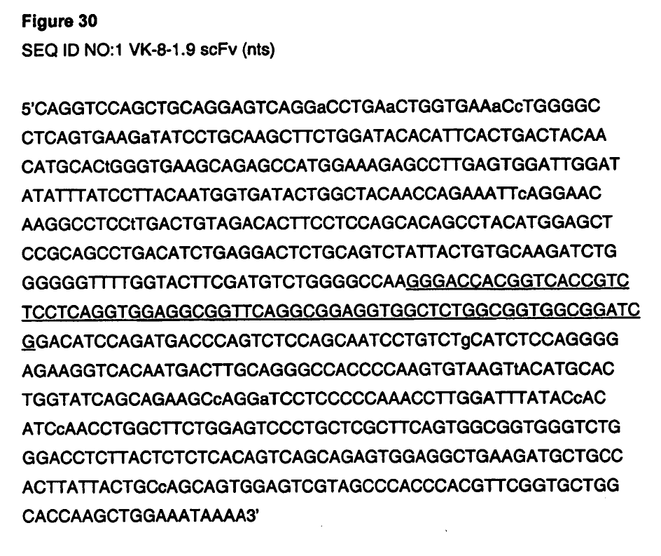

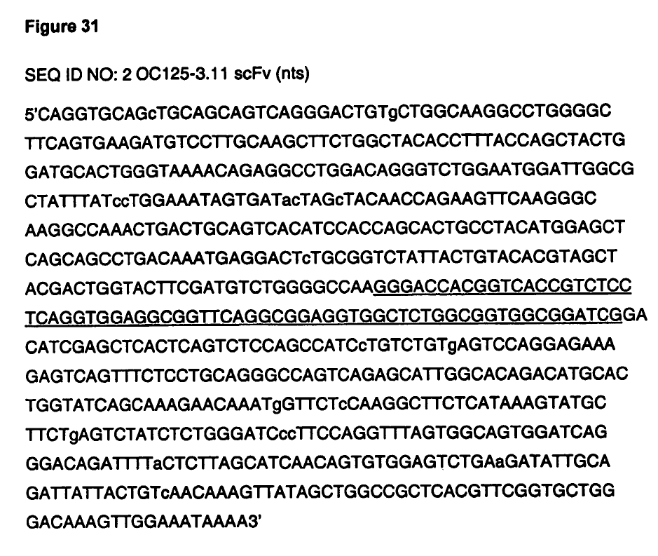

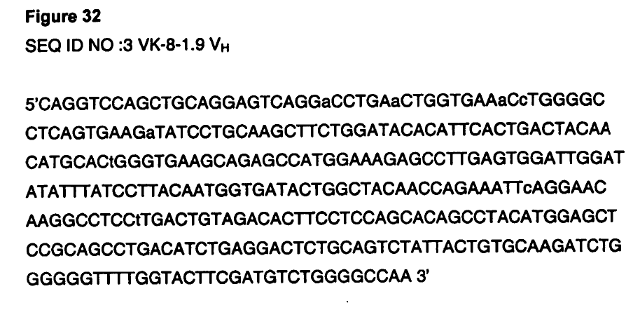

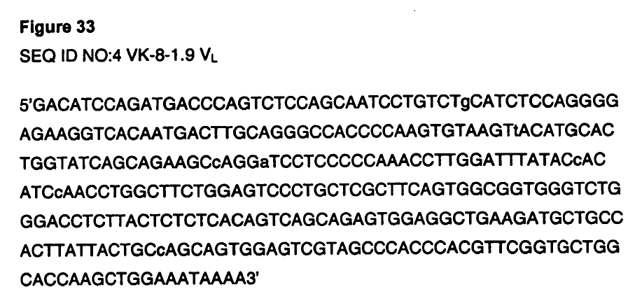

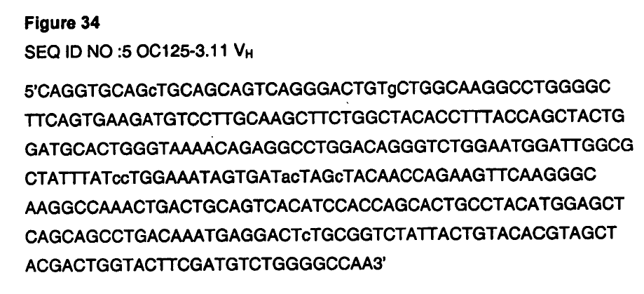

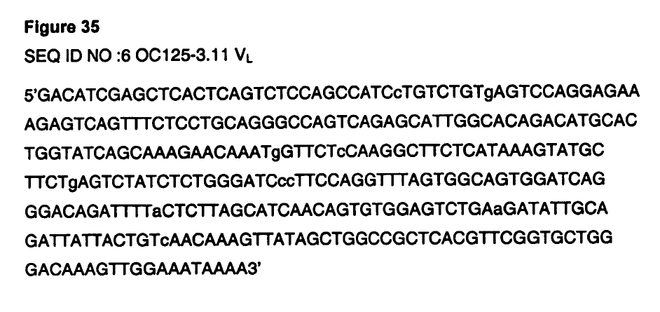

The inventors constructed single-chain antibody libraries derived from the OC125 and VK-8 hybridoma cell lines specific for CA125. The two scFv libraries were screened for CA125 binding activity by ELISA using commercially purified human CA125. ScFvs that bound to CA125 by ELISA, OC125-3.11 and VK-8-1.9, and one that did not bind, VK-8-4.5 were selected (FIG. 15A-B). The inventors hypothesized that if those scFvs (CA125 binders) once expressed intracellularly were localized to and retained within the ER or the Golgi then CA125 antigen would be entrapped during synthesis and thus be unable to localize at cell surface and interact with other intracellular and/or extracellular proteins to achieve its function(s). The scFvs were targeted to the ER or trans-median Golgi by sequence fusion with an Igκ secretion leader and a KDEL signal or fusion with the N-terminal 81 amino acids of human beta 1,4-galactosyltransferase, a protein resident of the trans-medial Golgi (47-49) in addition ot a c-myc tag at the C-terminus. Immunoprecipitation experiments showed that the anti-CA125 scFvs were immunoprecipitated with anti-c-myc antibody whereas only OC125-3.11 and VK-8-1.9 (both positive for CA125 binding) were co-immunoprecipitated using anti-CA125 OC125 and VK-8 MAbs (FIG. 5C-E). These results demonstrate that the OC125-3.11 and VK-8-1.9 anti-CA125 scFvs bind to CA125 in vitro.

### Example II

### Localization of anti-CA125 scFvs and Cell Surface Down-Regulation of CA125

Proper localization of the anti-CA125 scFvs of the invention in transient transfection of human ovarian cancer cells OVCAR-3 was demonstrated by immunofluorescence. Results obtained with ER-VK-8-1.9 and GOLGI-OC125-3.11 are shown in FIG. 16A. Immunofluorescence studies showed that cells expressing the Golgi-targeted OC125-3.11 or ER-targeted VK-8-1.9 lost expression of CA125 at the cell surface. However surrounding cells that did not express the scFvs (not transfected) were positive for CA125 at the cell surface. In addition, the presence of the ER-targeted VK-8-4.5, which did not bind CA125 by ELISA and immunoprecipitation experiments, did not affect expression of CA125 in the cells expressing this scFv. These results show that the expression and retention of ER- or GOLGI-targeted anti-CA125 scFvs results in CA125 down-regulation at the cell surface. Anti-CA125 scFvs act therefore act as potent negative modulators of CA125.

### Example III

### Consequences of CA125 Cell Surface Down-Regulation in Human Ovarian Cancer Cell Line NIH:OVCAR-3

To determine the effects of down-modulating CA125 expression at the cell surface, the inventors derived stable clones encoding the ER-targeted VK-8-1.9 and Golgi-OC125-3.11 (both positive for CA125 binding) and ER-VK-8-4.5 (negative for CA125 binding) scFvs in human ovarian cancer cell lines OVCAR-3 (high expresser of CA125), OV-90 (moderate expresser) and SKOV3ip1 cells (low expresser). Some of the OVCAR-3 clones have been already characterized for scFv and CA125 expression and all of the other clones (including in SKOV3ip1) have also been evaluated. Characterization of stable clones ER-VK-8-1.9#9 and ER-VK-8-4.5#12 is shown in FIG. 17. A dramatic decrease in CA125 expression at the cell surface was observed in stable clone ER-VK-8-1.9#9 (positive for CA125 binding) while CA125 expression was not affected in the clone ER-VK-8-4.5#12 (negative for CA125 binding) although the scFv in this clone was expressed at adequate levels (FIG. 17A). Similar results were obtained from FACS analysis (FIG. 17B). These results are consistent with results obtained previously from transient transfection experiments. Taken together these results demonstrate that the scFvs of the present invention act as specific negative modulators of CA125 and that the stable clones of the present invention provide unique tools.

Expression of E-Cadherin and αvβv Integrin at the Cell Surface

To further characterize the stable transfectants expressing the anti-CA125 scFvs the inventors evaluated the expression of E-cadherin and αvβv integrin at the cell surface for each stable transfectant. FIGS. 23 through 26 show that E-cadherin expression at the cell surface is not affected by the presence of the ER-VK-8-1.9 anti-CA125 scFv or the control ER-VK-8-4.5 demonstrating that E-cadherin expression is not modulated by CA125 levels. Expression of αvβv integrin at the cell surface of ER-VK-8-4.5 transfectant is also not affected by the expression of the scFv (FIG. 28). However, the stable transfectant expressing the ER-VK-8-1.9 anti-CA125 scFv shows a reduced level of αvβv integrin at the cell surface demonstrating that CA125 influences levels of αvβv integrin expression at the cell surface.

Cell Proliferation on Adhesive Support

In vitro growth kinetics of ER-VK-8-1.9 and ER-VK-8-4.5 clones was evaluated compared with that of the parental cell line using a XTT cell proliferation assay (50). Stable clone ER-VK-8-1.9#9 (positive for CA125 binding) grew faster than the ER-VK-8-4.5#12 (negative for CA125 binding) which grew at a rate similar to that of the parental OVCAR-3 cells (FIG. 18A). Stable clone ER-VK-8-1.9#9 seems to adhere faster to the plastic than OVCAR-3 cells or ER-VK-8-4.5#12 clone (not shown). These results show that loss of CA125 at the cell surface affects cell proliferation on adhesive support.

To further characterize this effect, clonogenic assays were performed to investigate the growth kinetics of each transfectant when seeded at low density on adhesive support. Increasing amounts of each transfectant, VK-8 KDEL/1:9#9 and KDEL/4:5#12, and the parental cell line NIH OVCAR-3 were seeded in 6-well plates and grown in the absence or the presence of doxycycline. Fourteen days later, cells were stained with Giemsa. The number of colonies was scored for each transfectant and plotted against the amount of cells seeded. FIG. 18.1A shows that no difference in the amounts of colonies formed by each transfectant, regardless of CA125 status, confirming that CA125 does not affect the ability of the cells to form colonies when seeded at low density on adhesive support. However, FIGS. 18.1B to D show that the size of colonies formed by transfectant VK-8-1.9#9 was much bigger and easier to visualize compared to those from transfectant VK-8-4.5#12 and the parental cell line NIH OVCAR-3 demonstrating that transfectant VK-8-1.9#9 grow at an increased rate compared to the controls. These results show that CA125 influences the growth rate of cells and does not however modulate the ability of the cells to form colonies on adhesive support when seeded at low density.

Sensitivity to Cisplatin

Consequently, sensitivity to cisplatin of the various stable transfectants was determined. Stable clone ER-VK-8-1.9#9, the ER-VK-8-4.5#12 and the parental cell lines were plated in triplicate in 96-well plates and exposed or not to increasing concentrations of cisplatin. Fours days later, cell proliferation was measured with a XTT assay. Percentage of survival was plotted against concentration of cisplatin. Curves represent results from 3 independent experiments (FIG. 20). Results showed that stable clone ER-VK-8-1.9#9 was more sensitive to cispaltin than control cells. IC50 were calculated and FIG. 21 shows that the stable clone ER-VK-8-1.9#9 is approximately 10-fold more sensitive to cisplatin than ER-VK-8-4.5#12 and the parental cell lines. Similar experiments were performed using taxol and results showed no difference between sensitivity of stable transfectant ER-VK-8-1.9#9, the ER-VK-8-4.5#12 and the parental cell lines confirming that the increased sensitivity of transfectant ER-VK-8-1.9#9 is linked to the increase in cell proliferation. These results demonstrate that CA125 influences cell proliferation and thereby controls the sensitivity to therapeutics drugs such as cisplatin.

Cell-Cycle Analysis

Cell proliferation is closely linked to rate of apoptosis and cell cycle. Therefore the cell cycle progression of each transfectant was investigated. DNA content of non-synchronized cells was stained with propidium iodide at various time points for approximately 48 hr and the percentage of cells in each phase of the cell cycle was determined by FACS analysis. FIG. 20.1A shows that the respective profiles of cell cycle progression of transfectant VK-8-1.9#9 and VK-8-4.5#12 differ significantly, mostly in the G2/M peak. FIG. 20.1B shows percentages of cells in each phase of the cell cycle. A dramatic increase in the number of cells in G2/M phase was obtained for transfectant VK-8-1.9#9 compared to the control demonstrating that the loss of CA125 results in an increase in cell cycle progression. These results therefore confirm that CA125 controls cell proliferation by modulating the rate of cell cycle progression.

Cell-Cell Interactions and Anchorage Independence

We also assessed the effect of CA125 cell surface down-regulation on the ability of the cells to mediate cell-cell interaction using a cell aggregation assay (51). Cell-cell interactions are measured by the ability of the cells to aggregate to each other and grow in clumps. Transfectant ER-VK-8-4.5#12 formed small aggregates and grew in small clumps similarly to the parental OVCAR-3 cells (FIG. 18B). In contrast, transfectant ER-VK-8-1.9#9 (positive for CA125 binding) did not form aggregate and only isolated single cells were observed. In addition, the single cells observed in this clone did not grow and looked as if they were dead or dying. These results show that loss of CA125 at the cell surface impairs the cells ability to mediate cell-cell interactions and to survive in anchorage-independent conditions.

Cell Migration

The inventors also determined the consequence of reducing CA125 expression levels at the cell surface on cell migration. We evaluated the cell motility of ER-VK-8-1.9#9 and ER-VK-8-4.5#12 stable transfectants and compared to the parental cell line using the wound or scratch assay (52). Cells were plated in 6-well plates and when confluent a wound was made in the monolayer using a razor blade. To distinguish between cell proliferation and cell migration, cell proliferation was inhibited with 20 mM hydroxyurea (53). Cells were incubated in the presence or the absence of FBS. In the absence of FBS none of the cells, neither the parental cells, were able to migrate and fill in the wound (not shown). This suggests that some factors present in the serum may be required for stimulating cell motility as showed by others in different tumor cell lines (54). However, in the presence of FBS, the only cells that did not migrate and fill in the wound were the cells from clone ER-VK-8-1.9#9 (positive for CA125 binding) (FIG. 18C). Cells from clone ER-VK-8-4.5#12 (negative for CA125 binding) migrated in a similar manner as the parental cells. These results show that CA125 affects cell migration of the OVCAR-3 cell line.

Tumor Growth

The inventors also determined whether the loss of CA125 expression at the cell surface affects the in vivo behaviour of human ovarian cancer cells in tumor-bearing mice subcutaneously or intraperitoneally. This was achieved by evaluating tumor growth, tumor burden, formation of ascites, presence of tumor cells in ascites, pattern of metastases spread and survival of mice. The tumorigenicity of each stable transfectant was also determined in nude mice. Stable clone ER-VK-8-1.9#9, the ER-VK-8-4.5#12 and the parental cell lines were inoculated subcutaneously in nude mice and tumors were allowed to grow for 6 weeks after which tumors were excised and tumor weight was measured. FIG. 18D shows that tumor derived from stable clone ER-VK-8-1.9#9 were significantly much smaller (if existant) than those from the ER-VK-8-4.5#12 and the parental cell lines demonstrating that CA125 influence the tumorigenic potential of ovarian cancer cells. When injected intraperitoneally, stable clone ER-VK-8-1.9#9 showed a significant slower growth, reduced volume of ascites, a decrease in the total number of viable tumor cells in suspension (in ascites) and therefore an overall increased in survival of mice (not shown).

These results taken together point to a role for CA125 in the pathogenesis of ovarian cancer by influencing tumor cell proliferation, tumor cell adhesion and migration, and in tumorigenesis. Results also show CA 125 controls sensitivity of these cells to chemotherapeutic drugs such as cisplatin.

### Experimental Procedures

Derivation of anti-CA 125 scFv constructs—The hybridoma cell line VK-8 which express a monoclonal antibody against CA 125 tumor antigen has been described previously and was kindly provided by K. O. Lloyd (Sloan-Kettering Memorial Cancer Center, New York, N.Y.) (18). Total mRNA was extracted from VK-8 hybridoma using the PolyA-rack kit from Promega. Total mRNA extracted from OC125 hybridoma cell line was kindly provided by R. C. Bast (MD Anderson Cancer Center, Houston, Tex.). ScFvs constructs were generated using the Recombinant Phage Antibody System (Amersham Pharmacia Biotech, Piscataway, N.J.) according to the manufacturer's instructions. Briefly, the variable heavy and light chains (V_{H} and V_{L}) were amplified from the cDNA by PCR using mouse variable region primers. The V_{H} and V_{L} DNA fragments were linked together by overlap extension PCR using a (Gly₄Ser)₃ linker to generate 750 bp scFv constructs with flanking SfiI and NotI sites. The scFv DNA fragments were inserted into SfiI/NotI sites of the prokaryotic expression vector pCANTAB5E from the Mouse ScFv Module (Amersham Pharmacia Biotech, Piscataway, N.J.). Screening of recombinant clones expressing a soluble scFv was accomplished by a colony lift assay as described previously (26).

Plasmids—The phagemid pCANTAB5E/scFv contains the anti-CA 125 scFvs encoding sequences under the control of the inducible lac promoter. The scFvs are expressed as fusion proteins with a peptide tag (Etag) at the C-terminus to allow easy immunodetection. The ER-targeting vector pSTCF.KDEL was previously described (27). The selected anti-CA 125 scFv DNA fragments were subcloned into the SfiI/NotI sites of pSTCF.KDEL just upstream and in frame with the c-myc tag/KDEL sequence. The pLTR retroviral expression vector was described in details elsewhere (28). The same anti-CA 125 scFv DNA fragments were subcloned into the pLTR.KDEL retroviral plasmid as SfiI/NotI sites after insertion of a SfiI/NotI containing polylinker at XhoI site of pLTR.KDEL. In this vector, expression of the scFvs is under the control of a tetracycline-inducible CMV promoter.

Binding analysis by ELISA—Periplasmic extracts were prepared as previously described (29). Elisa plates were coated with 10 μg of purified CA 125 tumor antigen in 200 μl carbonate buffer pH 9.6 and incubated overnight at 4°0 C. in a humidified chamber. Wells were washed 3 times with 200 μl PBS and blocked with 200 μl 2% BSA/PBS-0.05% Tween20 (for VK-8 derivative scFvs) or 200 μl 2% NFDM/PBS-0.1% Tween20 (for OC125 derivative scFvs) for 1 hr at room temperature in a humidified chamber. The plates were air dried, 100 μl of periplasmic extracts were added in 100 μl of blocking buffer and the plates were incubated at room temperature for 1 hr in humidified chamber. Parental monoclonal antibodies VK-8 and OC125 served as positive control and an anti-Bcl-2 scFv and periplasmic extract from bacteria without plasmid served as negative controls. The anti-Bcl-2 scFv was described previously (29). Plates were then washed 6 times with blocking buffer, wells were allowed to air dry, mouse anti-Etag antibody (Pharmacia Biotech, Piscataway, N.J.) was added 1:1000 in 200 μl 2% BSANPBS-0.05% Tween20 or 200□l 2% NFDM/PBS-0.1% Tween20 and plates were incubated for 1 h at 37° C. in a humidified chamber. Wells were washed again 6 times with PBS/0.5% Tween20 or PBS/0.1% Tween20 and anti-mouse mouse HRP-conjugated secondary antibody 1:2000 was added in 2% BSA/PBS-0.05% Tween20 or 2% NFDM/PBS-0.1% Tween20 and plates were incubated for another hour at 37° C. in a humidified chamber. The plates were subsequently washed 10 times with PBS/0.05% Tween20 or PBS/0.1% Tween20, air dried, 100 μl of HRP susbtrate were added and plates were incubated in darkness. After 15-30 min 100 μl of H₂SO₄ 1N were added in each well to stop the colorimetric reaction. The OD was read at 450 ηm in an ELISA plate reader.

Sequencing of DNA encoding anti-CA 125 scFvs—ScFv encoded sequences were determined from pCANTAB5E/scFv constructs using the scFv specific primers S1 and S6 from the pCANTAB5 sequencing primer set (Amersham Pharmacia Biotech, Piscataway, N.J.). Sequences were determined using the LI-COR automatic sequencing system (Bio S&T Inc., Lachine, QUE).

Cell lines—The human ovarian cancer NIH:OVCAR-3 and the green monkey kidney COS-7 cell lines were obtained from the American Type Culture Collection (Rockville, Md.). COS-7 cells were maintained in F12/DMEM (Biomedia, Drummondville, QUE) supplemented with 10% FBS (Biomedia, Drummondville, QUE), 2 mM L-glutamine (Biomedia, Drummondville, QUE), 100 units/ml penicillin (Cie, city, state) and 100 μg/ml streptomycin. NIH:OVCAR-3 cells were grown in RPMI 1640 (Biomedia, Drummondville, QUE) supplemented with 20% FBS (Biomedia, Drummondville, QUE), 2 mM L-glutamine (Biomedia, Drummondville, QUE), 100 units/ml penicillin, 100 μg/ml streptomycin and 10 μg/ml insulin. Both cell lines were maintained at 37° C. in a humidified 5% CO₂ incubator.

Immunoblot analysis—Periplasmic extracts (equal volume), cell lysates (equal amounts of proteins) or immunoprecipitates were submitted to SDS-PAGE electrophoresis (12%) and transferred onto PVDF membrane. The membranes were probed with either an anti-Etag antibody (Amersham Pharmacia Biotech, Piscataway, N.J.) for periplasmic extracts or the anti-c-myc 9E10 antibody (Cie, city, state) for cell lysates. A HRP-conjugated rabbit anti-mouse antibody (Jackson ImmunoResearch, West Grove, Pa.) was used at 1:10,000. The immunoblots were developed with chemiluminescence using commercially available ECL system according to the manufacturer's instruction.

Immunoprecipitation—NIH-OVCAR-3 cells were transiently transfected with the various scFv constructs using commercially available FuGene 6 transfection agent using the manufacturer's instruction. Forty-eight hours later, cell were collected and lysed on ice in NP40 buffer (0.5% NP-40, 0.5% sodium deoxycholate, 0.1% SDS, 100 mM NaCl, 1 mM EDTA, 20 mM Tris-HCl pH 8.0). Protein concentration was measured by the Bradford method using the Bio-Rad protein assay according to the instruction provided by the manufacturer. Three hundreds micrograms of total proteins were incubated with 1 ug of polyclonal anti-c-myc A-14 antibody (Santa Cruz), mouse anti-CA125 OC125 antibody or mouse anti-CA125 VK-8 antibodies for 1 hr on ice without shaking. Thirty microliters of NP40 buffer-prewashed protein A-sepharose beads (anti-c-myc) or protein-agarose beads were added, slowly shaked for 1 hr at 4° C. and spun down at 5000 rpm for 5 min. The pellets were washed 3 times in NP40 buffer and finally resuspended in 40 μl of NP40 buffer and boiled for 5 min. Agarose or sepharose beads were pelleted and supernatants were analyzed by immunoblots for the presence of the various scFvs.

Stable cell lines expressing anti-CA 125 scFvs—The stable cell lines OVCAR3-1.9#9, OVCAR3-4.5#12 and OVCAR3-GFP were generated by stable transfection of pLTR.KDEL-anti-CA 125 scFv#1.9, 2.7, 4.5 and GFP respectively. Stable clones were selected under lug/ml blasticidin exposure. Stable transfection of NIH:OVCAR-3 cells was performed using commercially available FuGene6 transfection reagent following manufacturer's instructions.

Immunofluorescence—Transiently transfected NIH-OVCAR-3 cells or stable NIH:OVCAR-3 clones expressing the various anti-CA125 scFvs were grown on glass slide until a 50% confluence was reached. Glass slides were then washed in cold PBS and cells fixed in ice-cold methanol for 10 min at −20° C. Glass slides were next rinsed 5 min in cold PBS and permeabilized in PBS containing 0.1% Triton X-100 for 5 min on ice and rinsed again in PBS. Slides were blocked in XmMPBS/2% BSA on ice for 45 min and then incubated with primary antibodies in blocking buffer at room temperature for 1 hr. Slides were next washed 3 times in cold PBS, incubated for 30 min at room temperature with anti-mouse or anti-rabbit secondary antibodies coupled to Texas Red or Oregon green (1:1000), washed with PBS and mounted for visualization by fluorescence microscopy. Expression of the various anti-CA125 scFvs and CA125 was detected using anti-c-myc antibody (9E10, 1:500) and OC125 antibody (1:500). Localization of the scFvs was determined by comparing their pattern of expression with that of calreticulin and ADP-ribosylation factor for ER and Golgi localization using anti-calreticulin antibody (1:10000) anti-calreticulin and anti-ADP-ribosylation factor (1:400).

Flow cytometry analysis—Expression levels of CA125 and the anti-CA125 scFvs in the various NIH:OVCAR-3 stable clones were analyzed by FACS. One million PBS/EDTA-treated cells were fixed in 2% para-formaldehyde for 20 min at room temperature. Cells were next permeabilized with 0.1% saponine for 20 min at room temperature and incubated with primary antibodies in 2% BSA/PBS locking buffer for 45 min at room temperature. CA125 and scFv expression was determined using the mouse anti-CA125 M11 antibody (1:500) and the polyclonal anti-c-myc antibody A14 (1:500) respectively. Cells were next pelleted and incubated with anti-mouse-PE (1:1000) and anti-rabbit-FITC (1:500) secondary antibodies in blocking buffer for 45 min at room temperature. Cells were pelleted, resuspended in an appropriate volume and analyzed using a FACS Scan cytometer (Becton Dickenson, Mississauga, Canada).

Although preferred embodiments of the present invention have been described in detail herein and illustrated in the accompanying drawings, it is to be understood that the invention is not limited to these precise embodiments and that various changes and modifications may be effected therein without departing from the scope or spirit of the present invention.

### REFERENCES

1. Bast R C Jr, Feeney M, Lazarus H. Nadler L M, et al. Reactivity of a monoclonal antibody with human ovarian carcinoma. J Clin Invest 1981, 68: 1331-1337.

2. Bast R C Jr, Klug T L, St-John E, Jenison E, et al. A radioimmunoassay using a monoclonal antibody to monitor the course of epithelial ovarian cancer. N Engl J Med 1983, 309: 883-887.

3. Canney P A, Moore M, Wilkinson P M and James R D. Ovarian cancer antigen CA125: a prospective clinical assessment of its role as a tumour marker. Br J Cancer 1984, 50: 765-769.

4. Vergote I B, Bormer O P and Abeler V M. Evaluation of serum CA 125 levels in the monitoring of ovarian cancer. Am J Obstet Gynecol 1987, 157, 88-92.

5. Niloff, J M, Knapp R C, Lavin P T, Malkasian G D, Berek J S, Mortel R, Whitney C, Zurawski V R and Bast R C Jr. The CA125 assay as a predictor of clinical recurrence in epithelial ovarian cancer. Am J Obstet Gynecol 1986, 155: 56-60.

6. Fish R G, Shelley M D, Maughan T, Rocker I et al. The clinical value of serum CA125 levels in ovarian cancer patients receiving platinum therapy. Eur J Cancer & Clin Oncol 1987, 23: 831-835.

7. Makar A P, Kristensen G B, Bormer O P, Trope C G. Is serum CA 125 at the time of relapse a prognostic indicator for further survival prognosis in patients with ovarian cancer?Gynecol Oncol 1993, 49: 73-79.

8. Rustin G J S, Nelstrop A E, McClean P, Brady M F et al. Defining response of ovarian carcinoma to initial chemotherapy according to serum CA 125. J Clin Oncol 1996, 14: 1545-1551.

9. De Los Frailes M T, Stark S, Jaeger W, Hoerauf A et al. Purification and characterization of the CA125 tumor-associated antigen from human ascites. Tumor Biol 1993, 14: 18-29.

10. Kobayashi H, Ida W, Terao T, Kawashima Y. Molecular characteristics of the CA 125 antigen produced by human endometrial epithelial cells: comparison between eutopic and heterotopic epithelial cells. Am J Obstet Gynecol 1993, 169: 725-730.

11. Matsuoka Y, Nakashima T, Endo K, Yoshida T, et al. Recognition of ovarian cancer antigen CA125 by murine monoclonal antibody produced by immunization of lung cancer cells. Cancer Res 1987, 6335-6340.

12. Nagata A, Hirota N, Sakai T, Fujimoto M and Komoda T. Molecular nature and possible prsence of a mebranous glycan-phosphatidylinositol anchor of CA125 antigen. Tumor Biol 1991, 12: 279-286.

13. Yu Y H, Schlossman D M, Harrison C L, Rhinehardt-Clark A, Soper J T, Klug T L, Zurawski V R and Bast R C Jr. Coexpression of different antigenic markers on moieties that bear CA 125 determinants. Cancer Res 1991, 51: 468-475.

14. Zurawski V R Jr, Davis H M, Finkler N J, Harrison C L, et al. Cancer Rev 1988, 11-12: 102-118.

15. Lloyd K O, Yin B W T, Kudryashov V. Isolation and characterization of ovarian cancer antigen CA125 using a new monoclonal antibody (VK-8): identification as a mucin-type molecule. Int J Cancer 1997, 71: 842-850.

16. Yin B W T. and Lloyd K O. Molecular cloning of the CA125 ovarian cancer antigen. J. Biol. Chem. 2001 276: 27371-27375.

17. O'Brien T J, Beard J B, Underwood L J, Dennis R A, Santin A D and York L. The CA125 gene: an extracellular superstructure dominated by repeat sequences. Tumor Biol 2001, 22: 348-366.

18. O'Brien T J, Beard J B, Underwood L J and Shigemasa K. The CA125 gene: a newly discovered extension of the glycosylated N-terminal domain doubles the size of this extracellular superstructure. Tumor Biol 2002, 23: 154-169.

19. Yin B W T, Dnistrian A. and Lloyd, K O. Ovarian cancer antigen CA125 is encoded by the MUC16 mucin gene. Int. J. Cancer 2002, 98: 737-740.

20. Kabawat S E, Bast R C Jr, Welch W R, Knapp R C and Colvin R B. Tissue distribution of a coelomic-epithelium-related antigen recognized by the monoclonal OC125. Am J Clin Pathol 1983, 79: 98-104.

21. Bischof P, Tseng L, Brioschii P A, Herman W L. Cancer antigen 125 is produced by human endometrial stromal cells. Hum Reprod 1986, 1: 423-426.

22. Karlan B Y, Amin W, Casper S E, Littlefield B A. Hormonal regulation of CA125 tumor marker expression in human ovarian carcinoma cells: inhibition by glucocorticoids. Cancer Res 1988, 48:3502-3506.

23. Brumsted J R, McBean J H, Deaton J L, Gibson M. CA-125 secretion by luteal phase endometrium in vitro. Hum Reprod 1990, 5: 682-684.

24. Marth C, Lang T, Kota A, Mayer I and Daxenbichier G. Transforming growth factor-beta and ovarian carcinoma cells: regulation of proliferation and surface antigen expression. Cancer Lett 1990, 51: 221-225.

25. Kurachi H, Adachi H, Morishige K, Adachi K, Takeda T, Homma H, Yamamoto T and Miyake A. Transforming growth factor-alpha promoters tumor markers secretion from human ovarian cancers in vitro. Cancer 1996, 78: 1049-54.

26. Konishi I, Fendrick J L, Parmley T H, Quirk J G and O'Brien T J. Epidermal growth factor enhances secretion of the ovarian tumor-associated cancer antigen CA125 from the human amnion WISH cell line. J Soc Gynecol Invest 1994, 1: 89-96.

27. Ishiwata I, Ishiwata C, Nazawa S, Ishiwata H. CA125 production by gynecologic tumors in vitro and its modulation induced by dibutyl cyclic adenosine monophosphate. Asia Oceania J Obstet Gynecol 1986, 12: 285-290.

28. Bonfrer J M, Linders T C, Hageman P C, Hilkens J G, Korse C M and Molthoff C F M. Effect of paclitaxel (taxol) on CA125 expression and release by ovarian cancer cell lines. Tumor Biol 1997, 18: 232-240.

29. Marth C, Zeimet A G, Widschwendter M, Ludescher C, Kaern J, Tropé C, Gastl G, Daxenbichler G and Dapunt O. Paclitaxel and docetaxel-dependent activation of CA125 expression in human ovarian carcinoma cells. Cancer Res 1997, 57: 3818-3822.

30. Konishi I, Fendrick J L, Parmley T H, Quirk J G Jr and O'Brien T J. Epidermal growth factor enhances secretion of the ovarian tumor-associated cancer antigen CA125 from the human amnion WISH cell line. J Soc Gynecol Invest 1993, 1: 89-96.

31. Fendrick J L, Konishi I, Parmley T H, Quirk J G and O'Brien T J. CA125 phosphorylation is associated with its secretion from the WISH human amnion cell line. Tumor Biol. 1997, 18: 278-289.

32. www.cbs.dtu.dk/cai-bin/

33. Marth C, Zeimeth A G, Widschwendter and Daxenbichler G. (1998) Regulation of CA125 expression in cultures human carcinoma cells. Int J Biol Markers 13(4): 207-209.

34. Auersperg N, Pan J, Grove B D, Peterson T, Fisher J, Maines-Bandiera S, Somasiri S and Roskelley C D. E-cadherin induces mesenchymal-to-epithelial transition in human ovarian surface epithelium. Proc. Natl. Acad. Sci. USA 1999, 96: 6249-6254.

35. Gendler S J and Spicer A P. Epithelial mucin genes. Annu Rev Physiol. 1995, 57: 607-634.

36. Cao Y, Blohm D, Ghadimi B M, Stosiek P, Xing P X and Karsten U. Mucins (MUC1 and MUC3) of gastrointestinal and breast epithelia reveal different and heterogenous tumr-associated aberrations in glycosylation. J Histochem Cytochem 1997, 45: 1547-57.

37. Hilkens J, Vos H L, Wesseling J, Boer M, Storm J, van der Valk S, Calafat J and Patriarca C. Is episialin/MUC1 involved in breast cancer progression? Cancer Lett 1995, 90: 27-33.

38. Spicer A P, Rowse G J, Lindner T K, Gendler S J. Delayed mammary tumor progression in Muc-1 null mice. J Biol Chem 1995, 270: 30090-30101.

39. Schroeder J A, Thompson M C, Mockensturm Gardner M, and Gendler S J. Transgenic MUC1 interacts with epidermal growth factor receptor and correlates with mitogen-activated protein kinase activation in the mouse mammary gland. J Biol Chem 2001, 276: 13057-13064.

40. Pandey P, Kharbanda S and Kufe D. Association of the DF3/MUC1 breast cancer antigen with Grb2 and the SOS/Ras exchange protein. Cancer Res 1995, 55: 4000-4003.

41. Suwa T, Honda Y, Makiguchi Y, Takahashi T, Itoh F, Adachi M, Hareyama M and Imai K. Increased invasiveness of MUC1 and cDNA-transfected human gastric cancer MKN74 cells. Int J Cancer 1998, 76: 377-382.

42. Yamamoto M, Bharti L Y, Kufe D. Interaction of the DF3/MUC1 breast carcinoma-associated antigen and beat-catenin in cell adhesion. J Biol Chem 1997, 272: 12492-12494.

43. Bharti L Y, Chen D, Gong J and Kufe D. Interaction of Glycogen Synthase Kinase 3□ with the DF3/MUC1 carcinoma-associated antigen and □catenin. Mol Cell Biol 1998, 18: 7216-7224.

44. Regimbald L H, Pilarski L M, Longenecker B M, Reddish M A, Zimmerman G and Hugh J C. The breast mucin MUC1 as a novel adhesion ligand for endothelial intercellular adhesion molecule 1 in breast cancer. Cancer Res 1996, 56: 4244-4249.

45. Kam J L, Regimbald L H, Hilgers J H, Hoffman P, Krantz M J, Longenecker B M and Hugh J C. MUC1 synthetic peptide inhibition of intercellular adhesion molecule-1 and MUC1 binding requires six tandem repeats. Cancer Res 1998, 58, 5577-5581.

46. Moniaux N, Escande F, Porchet N, Aubert J-P and Batra S. Structural organization and classification of the human mucin genes. Frontiers in bioscience 2001, 6: 1192-1206.

47. Llopis J, McCaffrey J M, Miyawaki A, Farquhar M G and Tsien R Y. Measurement of cytosolic, mitochondrial, and Golgi pH in single living cells with green fluorescent proteins. Proc Natl Acad Sci USA 1998, 95: 6803-6808.

48. Yamaguchi N and Fukuda M N. Golgi retention mechanism of beta-1,4-galactosyltransferase. Membrane-spanning domain-dependent homodimerization and association with alpha- and beta-tubulins. J Biol Chem 1995, 270: 12170-12176.

49. Gleeson P A, Teasdale R D and Burke J. Targeting of proteins to the Golgi apparatus. Glycoconjugate J 1994, 11: 381-394.

50. Hindshaw V S, Olsen C W, Dybdahl-Sissoko N and Evans D. Apoptosis: a mechanism of cell killing by influenza A and B viruses. J Virol 1994, 68: 3667-3673.

51. Boterberg T, Bracke M E, Bruyneel E A and Mareel M M. Cell aggreagation assays. In: Metastasis Research Protocols. Analysis of cell behavior in vitro and in vivo. Ed. S A Brooks and U Schumacher. Humana Press (2001) pp 33-45.

52. Niinaka Y, Haga A and Raz A. Quantification of cell motility. In: Metastasis Research Protocols. Analysis of cell behavior in vitro and in vivo. Ed. S A Brooks and U Schumacher. Humana Press (2001) pp 55-60.

53. Nurugesan G, Sa G and Fox P L. High-density lipoprotein stimulated endothelial cell movement by a mechanism distinct from basic fibroblast growth factor. Circ Res 1996, 74: 1149-1156.

54. Erdel M. Speiss E, Trifz G, Boxberger H J and Ebert W. Cell interactions and motility in human lung tumor cell lines HS-24 and SB-3 under the influence of extracellular matrix components and protease inhibitors. Anticancer Res 1992, 12: 349-360.

55. Elbashir S M, Harborth J, Lendeckel W, Yalcin A, Weber K and Tuschl T. Duplexes of 21-nucleotide RNAs mediate RNA interference in cultures mammalian cells. Nature 2001, 411: 494-498.

56. Elbashir S M, Lendeckel W and Tuschl T. RNA interference is mediated by 21- and 22-nucleotide RNAs. Genes and Dev 2001, 15: 188-200.

57. Elbashir S M, Martinez J, Patkaniowaks A, Lendeckel W and Tuschl T. Functional anatomy of siRNAs for mediating efficient RNAi in Drosophila melanogaster embryo lysate. Embo J 2001, 20: 6877-6888.

58. Harborth J, Elbashir S M, Bechert K, Tuschl T and Weber K. Identification of essential genes in cultured mammalian cells using small interfering RNAs. J Cell Sci 2001, 114: 4557-4565.

59. Paul C P, Good P D, Winer I and Engelke D R. Effective expression of small interfering RNA in human cells. Nature Biotech 2001, 29: 505-508.

60. Miyagishi M and Taira K. U6-promoter-driven siRNAs with four uridine 3′ overhangs efficiently suppress targeted gene expression in mammalian cells. Nature Biotech 2001, 29: 497-500.

61. Tuschl T. Expanding small RNA interference. Nature Biotech 2001, 20: 446-448.

62. Provencher D M, Lounis H, Champoux L, Tétreault M, Manderson E, Wang J C, Eydoux P, Savoie R, Tonin P N and Mes-Masson A-M. Characterization of four novel epithelial ovarian cancer cell lines. In Vitro Cell Dev Biol 2002, 36: 357-361.

63. Hamilton T C. Young R C. Louie K G. Behrens B C. McKoy W M. Grotzinger K R. Ozols R F. Characterization of a xenograft model of human ovarian carcinoma which produces ascites and intraabdominal carcinomatosis in mice. Cancer Res 1984, 44:5286-90.

64. Yu D. Wolf J K. Scanlon M. Price J E. Hung M C. Enhanced c-erbB-2/neu expression in human ovarian cancer cells correlates with more severe malignancy that can be suppressed by E1A. Cancer Res 1993, 53:891-8.

65. Piché A, Grim J, Rancourt C, Gomez-Navarro J, Reed J C and Curiel D T. Modulation of Bcl-2 protein levels by an intracellular anti-Bcl-2 single-chain antibody increases drug-induced cytotoxicity in the breast cancer cell line MCF-7. Cancer Res 1998, 58: 2134-2140.

66. Frankel A, Rosen K, Filmus J and Kerbel R S. Induction of anoikis and suppression of human ovarian tumor growth in vivo by down-regulation of Bcl-XL. Cancer Res 2001, 61: 4837-4841.

67. Grossman J. Molecular mechanisms of “detachment-induced apoptosis-Anoikis”. Apoptosis 2002, 7: 247-260.

68. Bank H L. Rapid assessment of islet viability with acridine orange and propidium iodide. In Vitro Cell Dev Biol 1988, 24: 266-273.

69. Kudoh M, Knee D A, Takayama S and Reed J C. Bag1 proteins regulate growth and survival of ZR-75-1 human breast cancer cells. Cancer Res 2002, 62: 1904-1909.

70. Cannistra S A, Kansas G S. Niloff J. DeFranzo B. Kim Y. Ottensmeier C. Binding of ovarian cancer cells to peritoneal mesothelium in vitro is partly mediated by CD44H. Cancer Res 1993, 53: 3830-3838.

71. Strobel T and Cannistra S A. Beta1-integrins partly mediate binding of ovarian cancer cells to peritoneal mesothelium in vitro. Gynecol Oncol 1999, 73: 362-367.

72. Carreiras F. Denoux Y. Staedel C. Lehmann M. Sichel F. Gauduchon P. Expression and localization of alpha v integrins and their ligand vitronectin in normal ovarian epithelium and in ovarian carcinoma. Gynecol Oncol 1996, 62: 260-267.

73. Wilson K E. Bartlett J M. Miller E P. Smyth J F. Mullen P. Miller W R. Langdon S P. Regulation and function of the extracellular matrix protein tenascin-C in ovarian cancer cell lines. Br J Cancer 1999, 80: 685-692.

74. Erickson C A. Cell migration in the embryo and adult organism. Curr Opin Cell Biol 1990, 2: 67-74.

75. Stoker M and Gherardi E. Regulation of cell movement: the motogernic cytokines. Biochem Biophys Acta 1991, 1072: 81-102.

76. Liotta L A, Mandler R, Murano Gm Katz D A, Gordon R K, Chiang P K and Schiffman E. Tumor cell autocrine factor. Proc Natl Acad Sci USA 1986, 83: 3302-3306.

77. Scotton C J, Wilson J L, Milliken D, Stamp G and Balkwill F R. Epithelial cancer cell migration: a role for chemokine receptors? Cancer Res 2001, 61: 4961-4965.

78. Venkatakrishnan G, Salgia R and Groopman J E. Chemokine receptors CXCR-1/2 activate mitogen-activated protein kinase via the epidermal growth factor receptor in ovarian cancer cells. J Biol Chem 2000, 275: 6868-6875.

79. Rosano L, Varmi M, Salani D, DiCastro V, Spinella F, Natali P G and Bagnato A. Endothelin-1 induces tumor ptoeinase activation and invasiveness of ovarian carcinoma cells. Cancer Res 2001, 61: 8340-8346.

80. Ellerbroek S M, Halbleib J M, Benavidez M, Warmka J K, Wattenberg E V, Stack M S and Hudson L. Phosphatidylinositol 3-kinase activity in epidermal growth factor-stimulated matrix metalloproteinase-9 production and cell surface association. Cancer Res 2001, 61: 1855-1861.

81. Lu J, Xiao Y, Baudhuin L M, Hong G and Xu Y. Role of ether-kinked lysophosphatidic acids in ovarian cancer cells. J Lipid Res 2002, 43: 463-476.

82. Bourguignon L Y W, Zhu H, Shao L and Chen Y-W. CD44 interaction with c-Src kinase promotes cortactin-mediated cytoskeleton function and hyaluronic acid-dependent ovarian tumor cell migration. J Biol Chem 2001, 276: 7327-7336.

83. Naylor M S, Stamp G W H, Davies B and Balkwill F R. Expression and activity of MMPs and their regulators in ovarian cancers. Int J Cancer 1994, 58: 50-56.

84. Niebdala M J, Crickard K and Bernacki R J. In vitro degradation of extracellular matrix by human ovarian cancer cells. Clin Exp Metastasis 1987, 181-197.

85. Stack M S, Ellerbroeck S M and Fishman D A. The role of proteolytic enzyme in the pathology of epithelial ovarian carcinoma. Int J Cancer 1998, 12: 569-576.

86. Afzal S, Lalani E N, Foulkes W D, Boyce B, Tickle S, Cardillo M R, Baker T, Pignatelli M and Stamp G W H. Matrix metalloproteinase-2 and tissue inhibitor of metalloproteinase-2 expression and synthetic matrix metalloproteinase-2 inhibitor binding in ovarian carcinomas and tumor cell lines. Lab Invest 1996, 74: 406-421.

87. Sternlicht M D, Bissel M J and Werb Z. The matrix metalloproteinase stromelysin-1 acts as a natural mammary tumor promoter. Oncogene 2000, 19: 1102-1113.

88. Wilson C L and Matrisian L M. Matrilysin: an epithelial matrix metalloproteinase with potentially novel functions. Int J Biochem 1996, 28: 123-136.

89. Pendas A M, Uria J A, Jimenez M G, Balbin M, Freije J P and Lopez-Otin C. An overview of collagenase-3 expression in malignant tumors and analysis of its potential value as a taget in antitumor therapies. Clin Chim Acta 2000, 291; 137-155.

90. Chambers S K, Gertz R E, Ivins C M and Kacinski B M. The significance of urokinase-type plasminogen activator, its inhibition, and its receptor in ascites of patiens with epithelial ovarian cancer. Cancer (Phila.) 1995, 75: 1627-1633.

91. Ellerbroek S M, Fishman D A, Kearns A S, Bafetti L M and Stack M S. Ovarian carcinoma cell regulation of matrix metalloproteinase-2 and membrane-type 1 matrix metalloproteinase through □1 integrin. Cancer Res 1999, 59: 1635-1641.

92. Fishman D A, Bafetti L M and Stack M S. Membrane-type metalloproteinase expression and matrix metalloproteinase-2 activation in primary human ovarian epithelial carcinoma cells. Invasion Metastasis 1996, 16: 150-159.

93. Leber T M and Balkwill F R. Zymography: a single-step staining method for quantitation of proteolytic activity on substrate gels. Analytical Biochem 1997, 249: 24-28.

94. Shibata K. Kikkawa F. Nawa A. Tamakoshi K. Suganuma N. Tomoda Y. Increased matrix metalloproteinase-9 activity in human ovarian cancer cells cultured with conditioned medium from human peritoneal tissue. Clin Exp Metastasis 1997, 15: 612-619.

95. Cruet S, Carreiras F, Staedel C and Guaduchon P. Molécules d'adhérence et proteases dans les cancer épithéliaux de l'ovaire. Médecine/Science 1999, 15: 645-654.

96. Yu J S, Sena-Esteves M, Paulus W, Breakefidle So and Reeves S A. Retroviral delivery and tetracycline-dependent expression of IL-1B-converting enzyme (ICE) in a rat glioma model provides controlled induction of apoptotic death in tumor cells. Cancer Res 1996, 56: 5423-5427.

97. Dimri G P, Lee X, Basile G, Agosta M, Scott G, Roskelley C, Medrano E E, Linsken M, Rubelj I, et al. A biomarker that identifies senescent human cells in culture and in aging skin in vivo. Proc Natl Acad Sci USA 1995, 92: 9363-9367.

98.Ozols R, Rubin, S C, Thomas G and Robboy S. Epihtelial ovarian cancer. In: Principles and practice of gynecologic oncology, Lippincott-Raven, 3^{rd} ed., pp. 981-1057, 2000.

99. James P, Halladay J and Craig E A. Genomic libraries and a host strain designed for highly efficient two-hybrid selection in yeast. Genetics 1996, 144: 1425-1436.

100. Tremblay A, Lamontagne B, Catala M, Yeung Y, Larose S, Good L and Abou Elela S. (2002) A physical interaction between Gar 1p and Rnt 1p is required for the nuclear import of H/ACA small RNA-asociated proteins. Mol Cell Biol 22: 4792-4802.

What is claimed is:

1. A modulator capable of negatively modulating a CA 125 tumor antigen in a mammalian cell.

2. The modulator according to claim 1, wherein it negatively modulates cell surface expression of CA 125 tumor antigen.

3. The modulator according to claim 1 or 2, wherein it sequesters CA 125 tumor antigen or a fragment thereof within an organelle of a mammalian cell.

4. The modulator according to claim 3, wherein the organelle is selected from the group consisting of the endoplasmic reticulum, the trans-golgi, the golgi, the mitochondrion, the cytoplasm and a cellular compartment.

5. The modulator according to any one of claims 1 to 4, wherein it is a single-chain antibody that specifically binds to CA 125 tumor antigen or a fragment thereof.

6. The modulator according to claim 5, wherein the single-chain antibody is derived from the group consisting of OC 125 monoclonal antibody and VK-8 monoclonal antibody.

7. The modulator according to claim 5 or 6, wherein the single-chain antibody comprises a fragment coded by at least one sequence of the group consisting of SEQ. ID NOS 1 to 6.

8. The modulator according to claim 7, wherein the single-chain antibody is coded by a sequence selected from the group consisting of SEQ. ID NOS 1 and 2.

9. A recombinant nucleic acid comprising at least one sequence selected from the group consisting of SEQ ID NOS 1 to 6.

10. A vector comprising a recombinant nucleic acid according to claim 10.

11. A host cell comprising at least one element selected from the group consisting of:
a modulator according to any one of claims 1 to 8;
a recombinant nucleic acid according to claim 9; and
a vector according to claim 10.

12. A pharmaceutical composition comprising a pharmaceutically acceptable carrier and at least one element selected from the group consisting of:
a modulator according to any one of claims 1 to 8;
a recombinant nucleic acid according to claim 9;
a vector according to claim 10; and
a host cell according to claim 11.

13. Use of at least one element selected from the group consisting of:
a modulator according to any one of claims 1 to 8;
a recombinant nucleic acid according to claim 9; and
a vector according to claim 10;
for negatively modulating a CA 125 tumor antigen in a mammalian cell.

14. Use of at least one element selected from the group consisting of:
a modulator according to any one of claims 1 to 8;
a recombinant nucleic acid according to claim 9;
a vector according to claim 10;
a host cell according to claim 11; and
a pharmaceutical composition according to claim 12;
for preventing and treating a CA 125-tumor-antigen-associated disease in a mammal.

15. A method of prevention or treatment of a CA 125-tumor-antigen-associated disease in a mammal comprising the step of administrating to said mammal at least one element selected from the group consisting of:
a modulator according to any one of claims 1 to 9;
a recombinant nucleic acid according to claim 10;
a vector according to claim 11; and
a host cell according to claim 12.

16. A method for negatively modulating a CA 125 tumor antigen in a mammalian cell comprising the step of introducing into said cell at least one element selected from the group consisting of:
a modulator according to any one of claims 1 to 9;
a recombinant nucleic acid according to claim 10; and
a vector according to claim 11.
